(19) United States
(12) Patent Application Publication
(10) Pub. No.: US 2022/0332759 A1
(43) Pub. Date: Oct. 20, 2022

(54) TARGETED PULMONARY DELIVERY COMPOSITIONS AND METHODS USING SAME

(71) Applicant: Rutgers, The State University of New Jersey, New Brunswick, NJ (US)

(72) Inventors: Renata Pasqualini, New Brunswick, NJ (US); Wadih Arap, New Brunswick, NJ (US); Daniela Staquicini, New Brunswick, NJ (US)

(21) Appl. No.: 17/765,379

(22) PCT Filed: Oct. 1, 2020
(86) PCT No.: PCT/US2020/053758

§ 371 Date: Mar. 30, 2022

Related U.S. Application Data
(60) Provisional application No. 62/910,998, filed on Oct. 4, 2019.

(51) Int. Cl. C07K 7/06; C12N 15/10; A61K 39/12

(52) U.S. Cl. CPC C07K 7/06; C12N 15/1037; A61K 39/12; A61K 38/00

(57) ABSTRACT

The present disclosure relates, in one aspect, to the identification of certain peptides that allow for transport of a solid particle across the air-blood barrier in the lungs. In certain embodiments, the solid particle comprises any sort of solid cargo to which the peptides contemplated in the disclosure can be attached.

### CROSS-REFERENCE TO RELATED APPLICATIONS

This application claims priority under 35 U.S.C. § 119(e) to U.S. Provisional Patent Application No. 62/910,998, filed Oct. 4, 2019, which is incorporated herein by reference in its entirety.

### BACKGROUND

Inhalation-based aerosol vaccination to achieve rapid immunization, especially in developing countries and disaster areas, is needle-free and, unlike oral therapies, are not subject to first-pass metabolism. The respiratory area of the lung tissue comprises over 90% of the total organ volume, the equivalent to about 80 square meters. The thin and highly permeable lung epithelium generally defines the selective permeability of molecules allowed to cross into the bloodstream. Small molecules, peptides or proteins such as insulin, and viral vaccines are the most suitable candidates for inhaled therapies. Lipophilic molecules are rapidly absorbed through the lungs, likely by passive diffusion across the plasma membrane, while hydrophilic molecules can be transported by specific cell receptors or via tight junctions.

In fact, the aerosol mode of phage-based vaccine introduction follows the route of many infections. Over the past two decades, major efforts focused on the optimization of a prototypical inhalation-based form of insulin. The current formulation approved by the FDA is still under evaluation in large population-based trials, as safety and/or efficacy concerns have hampered its broad commercial appeal. Most recently, aerosol-based vaccination platforms have gained particular attention for effective field protection against airborne pathogens such as tuberculosis, influenza, Ebola, and measles.

Aerosolization and pulmonary drug delivery improve drug bioavailability while reducing potential side-effects by achieving a more rapid onset of action. However, this route also poses many challenges, especially for systemic applications, restricting its use to respiratory diseases. Aerosolized therapies are generally assessed by monitoring the pharmacological endpoints in vivo. The development of new and efficient therapies is further hindered by the remarkable gap in knowledge about mechanisms of transport and the fate of aerosolized agents: the actual mechanisms of how inhaled particles interact with the air-blood barrier, the physicochemical changes in the aerosolized molecules in contact with the pulmonary surface, bioavailability, and finally the clearance process and removal of insoluble active compounds.

Thus, there is a need in the art for novel compositions that allow for pulmonary delivery of biologically active agents. In certain embodiments, such constructs could be used to promote targeted pulmonary vaccinations. The present disclosure addresses and satisfies this need.

### BRIEF DESCRIPTION OF THE DRAWINGS

The following detailed description of specific embodiments of the disclosure will be better understood when read in conjunction with the appended drawings. For the purpose of illustrating the disclosure, exemplary embodiments are shown in the drawings. It should be understood, however, that the disclosure is not limited to the precise arrangements and instrumentalities of the embodiments shown in the drawings.

FIGS. 1A-1F depict combinatorial aerosol selection of a phage display library and identification of a new ligand peptide-mediated transport. FIG. 1A: A random phage display library (CX8C) was administered via aerosol and recovered from the bloodstream at fixed time points up to six hours. FIG. 1B: Schematic of the four rounds of phage display library selection and time-to-collection for each round: R1 (60 min), R2 (30 min), R3 (10 min) and R4 (5 min). (C) Phage enrichment throughout the selection. The data are mean±SD (*P<0.05; *** P<0.001). FIG. 1D: Percentage of peptide motifs recovered from the fourth round of selections (R4). The cyclic peptides recovered with frequency equal or greater than 5% are: CAINSLSRKC, CAKSMGDIVC, CGRKQVESSC, and CRGKSAEGTC. Sixteen peptides with frequency less than 5% each were also identified. FIG. 1E: Transport of phage particles displaying the peptide motifs identified in R4. Insertless phage was used as a control. The data are mean±SD (*** P<0.001; n.s. stands for non-statistically significant). FIG. 1F: Transport of targeted CAKSMGDIVC-displaying phage particles and negative control insertless phage particles in vivo. The data are mean±SEM (*P<0.05; *** P<0.001).

FIGS. 2A-2D depict lung homeostasis remains normal upon peptide-mediated phage transport. FIG. 2A: Gross morphology and hematoxylin and eosin (H&E) staining of lung tissue sections from mice administered via aerosol with vehicle alone (PBS), negative control insertless phage particles, targeted CAKSMGDIVC-displaying phage particles, or LPS-dextran. FIG. 2B: Lung permeability, total proteins recovered from BALF, and infiltrating neutrophils were measured in mice administered via aerosol with targeted CAKSMGDIVC-displaying phage particles, negative controls (either insertless phage or vehicle alone), or the LPS injury model (positive control: LPS-dextran) (n=3 mice per group). Data are representative of three independent experiments. The bars represent mean±SEM (*** P<0.0001; ** P<0.01). FIG. 2C: Immunohistochemistry of phage overlay binding assays with targeted CAKSMGDIVC-displaying phage particles or control insertless phage particles in lung or control organ (pancreas) of mouse tissue sections. FIG. 2D: Relative quantification of the number of phage particles in the entire lung over time (*** P<0.001). Scale bar, 100 μm.

FIGS. 3A-3I depict identification and in vitro validation of α3β1 integrin as the receptor for the targeted CAKSMGDIVC-displaying phage particles. FIG. 3A: Targeted CAKSMGDIVC-displaying phage particles bind to human recombinant α3β1 integrin. BSA and insertless phage were used as negative controls (***P<0.001). FIG. 3B: Concentration-dependent inhibition of targeted CAKSMGDIVC-displaying phage particles binding to α3β1 integrin by its cognate synthetic peptide relative to an unrelated control peptide. FIG. 3C:

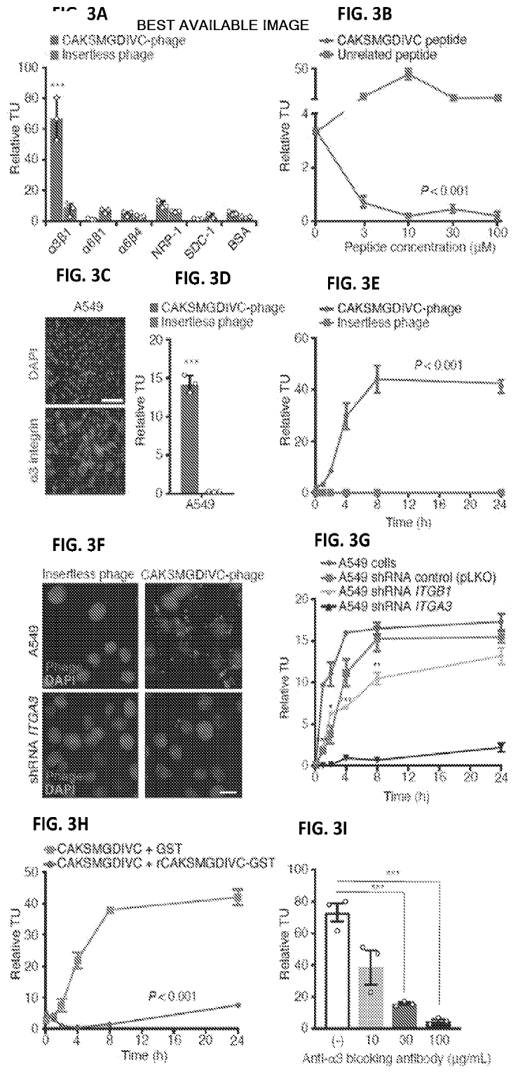

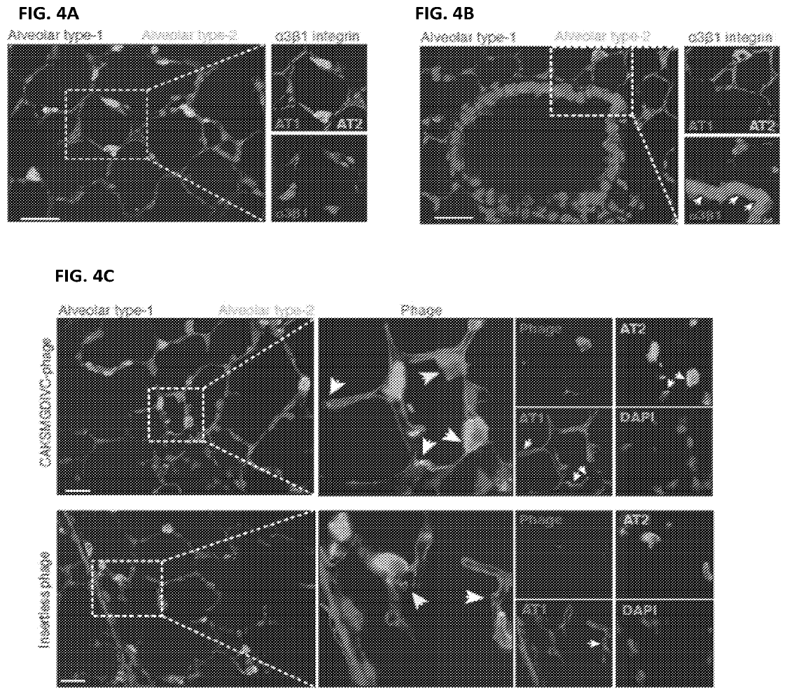

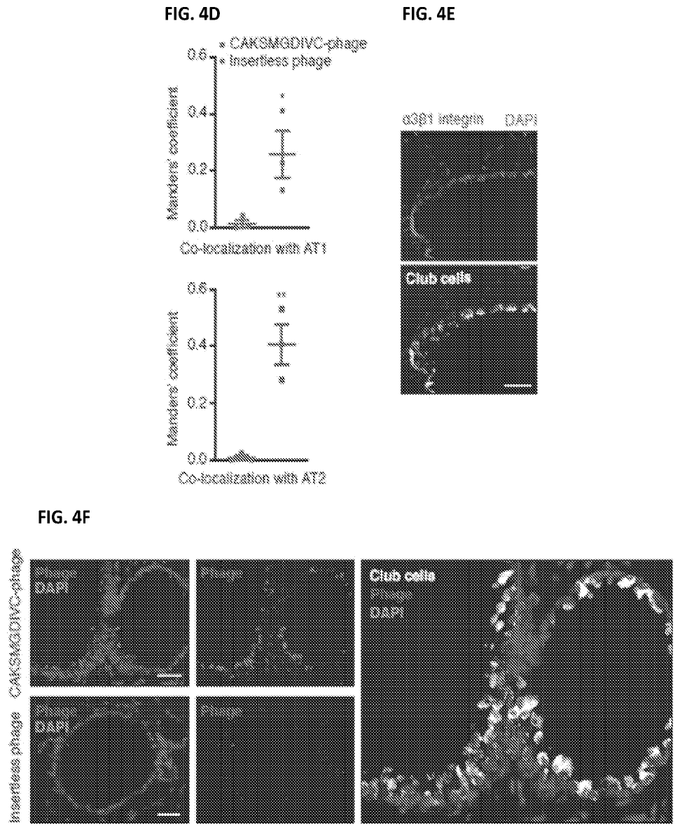

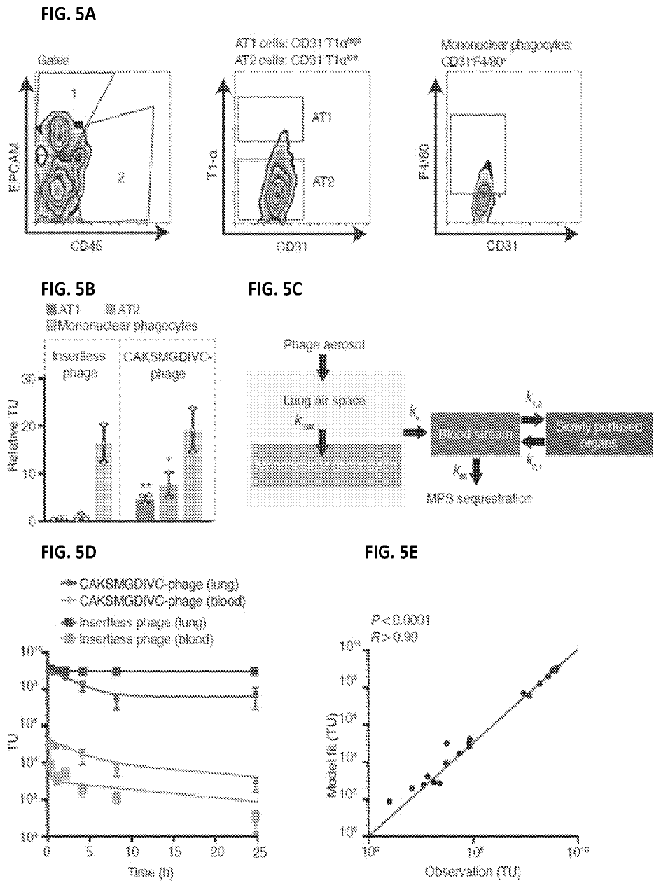

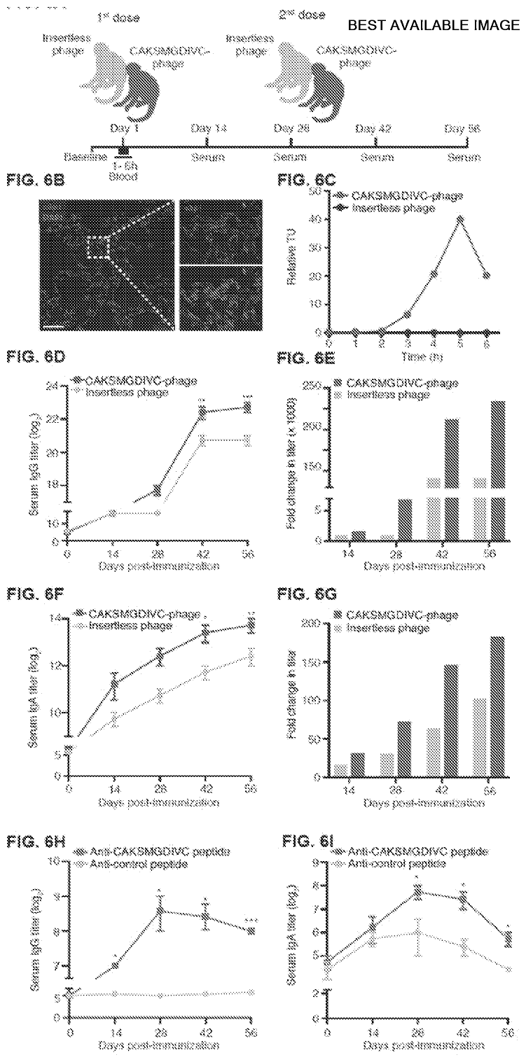

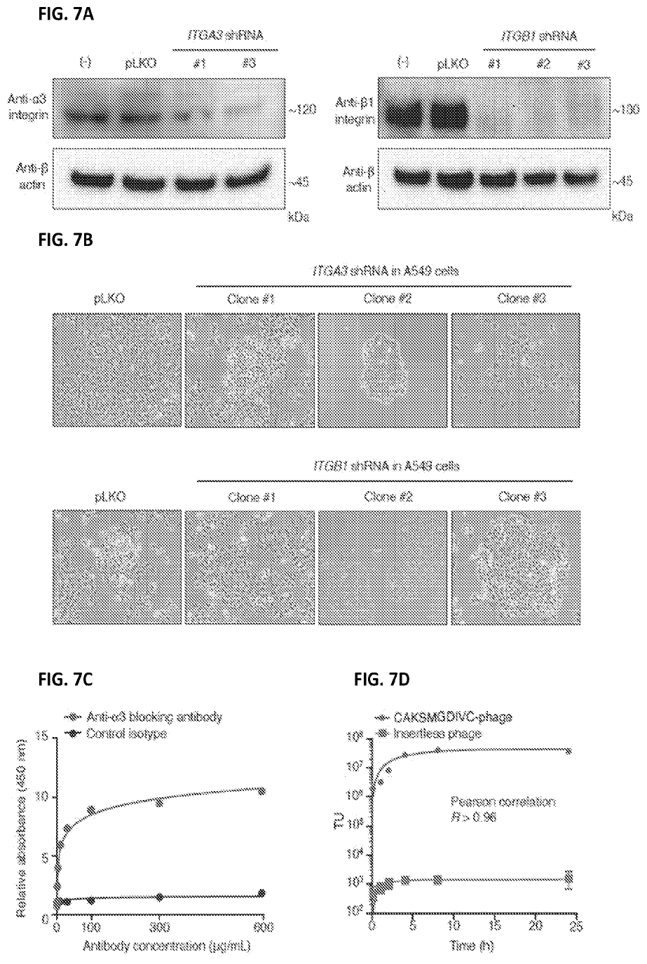

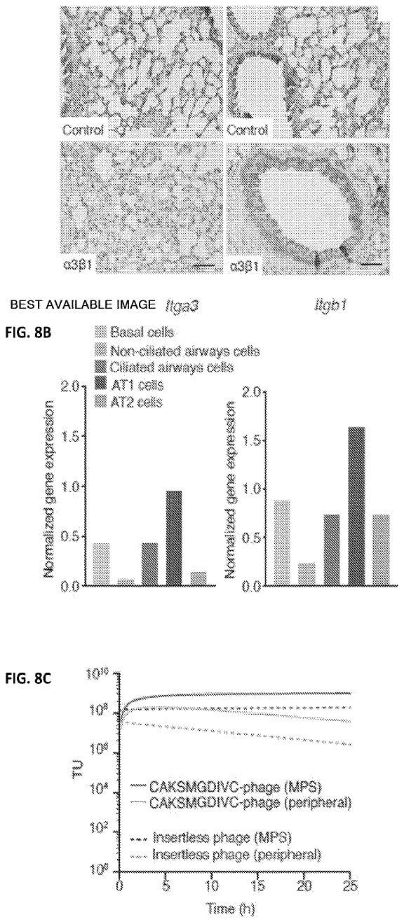

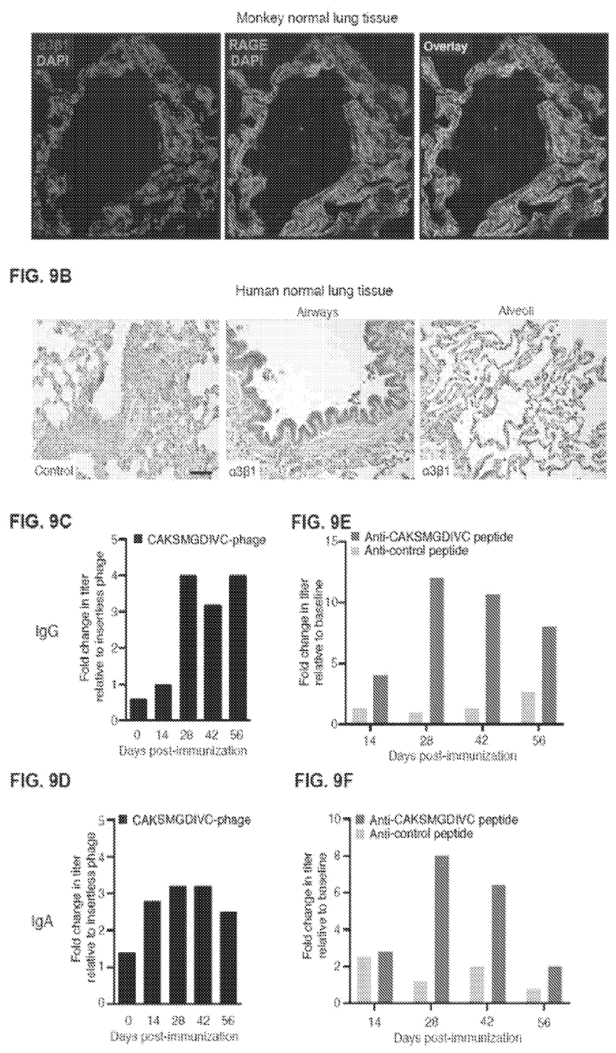

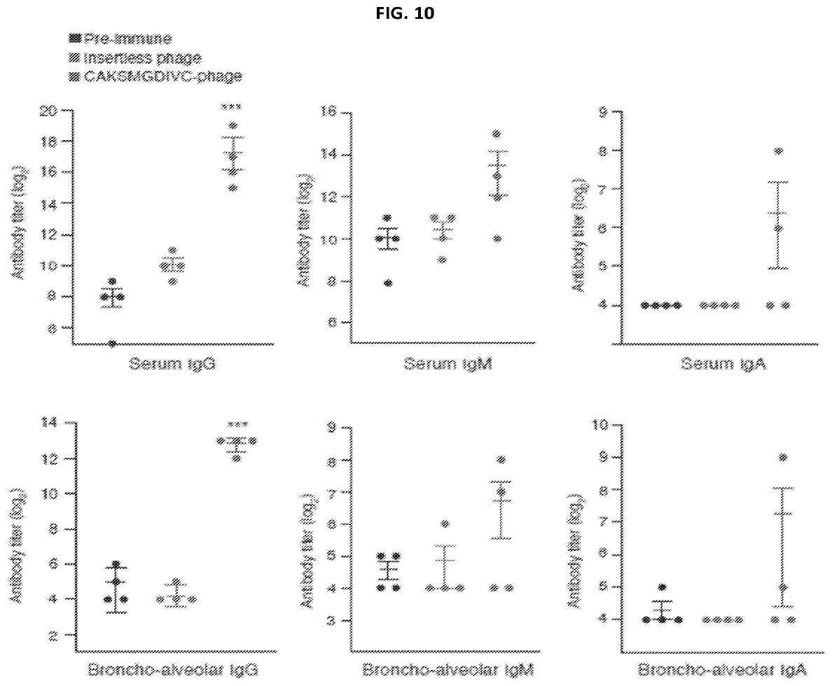

Immunofluorescence images of A549 cells stained with DAPI (blue) and anti-α3 chain antibody followed by a secondary antibody Cy3-conjugated (red) (scale bar, 100 μm). FIG. 3D: Phage binding on the surface of A549 cells (***P<0.001). FIG. 3E: Transport of targeted CAKSMGDIVC-displaying phage particles or control insertless phage particles through an A549 cell monolayer in Transwell assays. FIGS. 3F-3I: Phage internalization in A549 wild type cells and in A549 cells transduced with shRNA ITGA3 by immunofluorescence analysis with an anti-phage antibody followed by a secondary anti-Cy3-conjugated antibody (red) (scale bar, 50 μm). Targeted CAKSMGDIVC-displaying phage particles transport inhibition assays in Transwell systems: A549 cells silenced for the α3-chain (shRNA ITGA3) or (31-chain (shRNA ITGB1) (***P=0.001, *P=0.0417, ***P=0.008, **P=0.0136) (FIG. 3F) or with the recombinant proteins: GST (100 ng) (FIG. 3G), or CAKSMGDIVC-GST (100 ng) (FIG. 3H), or anti-α3 blocking antibody (FIG. 3I) at different concentrations for 1 h. No inhibitory effect was observed in the controls, wild type A549 cells or control cells transduced with the untargeted shRNA (pLKO) or with GST alone or the isotype control IgG antibody (***P<0.001).

FIGS. 4A-4F depict expression, localization, and binding of the ligand CAKSMGDIVC peptide to α3β1 integrins in lung tissue sections. FIGS. 4A-4B: Immunofluorescence of sectioned paraffin-embedded lung tissue sections. Alveolar epithelial cells type-1 (AT1) were stained with an anti-podoplanin antibody (purple), alveolar epithelial cells type-2 (AT2) were stained with anti-pro SPC antibody (green), anti-α3 chain antibody (red) and DAPI (blue). White arrows indicating the presence of α3β1 integrins in respiratory bronchioles. FIGS. 4A-B show the detection of α3β1 integrins in cells in the airways and alveolar regions of the lung, particularly in alveolar epithelial type-1 (AT1), alveolar epithelial type-2 (AT2) and cells of the respiratory bronchioles. Scale bar: 50 μm. FIG. 4C: Immunofluorescence analysis of lung tissue sections obtained from animals administered via aerosol with targeted CAKSMGDIVC-displaying phage particles or control insertless phage particles: AT1 cells (purple), AT2 cells (green), phage (red) and DAPI (blue) were imaged with a confocal microscope. White arrows show the presence of targeted CAKSMGDIVC-displaying phage particles or control insertless phage particles co-localizing with AT1 and AT2 cells. Yellow arrows show phage particles in alveoli macrophages. FIG. 4D: Manders' overlap coefficient for CAKSMGDIVC-displaying phage particles or control insertless phage particles in co-localization with alveolar epithelial AT1 or AT2 cells (*P=0.0439, **P=0.0053). FIG. 4E: Club cells were stained with an anti-CCSP antibody (white) and anti-α3 integrin chain antibody (red). DAPI (blue) was used to stain individual cell nuclei. Scale bar: 100 μm. FIG. 4F: Presence of targeted CAKSMGDIVC-displaying phage particles (red) or control insertless phage particles are shown co-localized with cells stained with an anti-CCSP antibody (white). Scale bar: 50 μm.

FIGS. 5A-5E depict that ligand CAKSMGDIVC peptide binds to alveolar epithelial AT1-, AT2-enriched cell populations and to club cells. FIG. 5A: Cell sorting by flow cytometry. Lung cells were isolated and purified from cohorts of animals (n=5) administered via aerosol with targeted CAKSMGDIVC-displaying phage particles or control insertless phage particles and stained with the following antibodies: anti-EPCAM, anti-CD45, anti-T1-α, anti-CD31, and anti-F4/80. FIG. 5B: The cells were gated based on their specific phenotype (gate 1:

CD31 and T1-α; gate 2: CD31 and F4/80), sorted, centrifuged and the amount of phage determined by counting TU. The data are representative of three independent experiments (**P=0.0053, *P=0.0375). FIG. 5C: Schematic of two-compartment pharmacokinetic model with extravascular (pulmonary) administration. The blood stream and rapidly perfused organs (central compartment) and slowly perfused organs (peripheral compartment). Mononuclear phagocyte system (MPS) sequestration refers to the clearance of phage particles by the MPS organs (liver, spleen). The lung, which is the site of administration, is compartmentalized into the lung airspace and mononuclear phagocytes. Aerosol phage particles from the lung airspace are either transported into the blood stream (central compartment) or are internalized into the macrophages. FIG. 5D: Fits of the pharmacokinetic model for targeted CAKSMGDIVC-displaying phage particles or control insertless phage particles. Data represents mean±SD (n=3). FIG. 5E: Pearson correlation coefficient R>0.99 showing strong correlation between observed data and model fits. Note: The y-axis is in log10 scale in FIGS. 5D-5E.

FIGS. 6A-6I depict that the intratracheal administration of CAKSMGDIVC-displaying phage particles elicits a robust and specific systemic antibody response in rhesus monkeys. FIG. 6A: Schematic of the intratracheal administration of targeted CAKSMGDIVC-displaying phage particles or control insertless phage particles in rhesus monkeys. Prior to the treatment, blood samples (baseline) were collected. Beginning at the time of the first dose (day 1), blood samples (1 mL) were collected hourly from 1 h up to 6 h. Over the course of the study, serum samples were collected every 14 days as indicated. FIG. 6B: Immunofluorescence analysis of α3β1 integrins (red) in the alveolar epithelial cells of lung tissue sections from rhesus monkeys. DAPI (blue) was used to stain individual cell nuclei. Scale bar:100 μm. FIG. 6C: Presence of phage particles in the blood stream of the rhesus monkeys administered with targeted CAKSMGDIVC-displaying phage particles. Phage load was determined by TU count. FIG. 6D: Titers of total purified phage-specific serum IgG antibodies were analyzed by ELISA in 96-well plates coated with 10¹⁰ phage particles per well (**P<0.01, ***P=0.0004). FIG. 6E: Fold change in titer of phage-specific IgG was calculated dividing the mean of antibody titer from each time point by the mean of antibody titer from the baseline. FIGS. 6F-6G: Total purified phage-specific serum IgA (FIG. 6F) and fold change in titer for IgA antibodies (FIG. 6G) were determined as described above (*P<0,05, **P<0.01). FIGS. 6H-6I: CAKSMGDIVC-specific IgG (FIG. 6H) and CAKSMGDIVC-specific IgA (FIG. 6I) were analyzed by ELISA in 96-well plates coated with synthetic CAKSMGDIVC peptide or with unrelated control peptide (*P<0,05, ***P<0.001). Note: The y-axis in log2 scale in FIGS. 6D, 6F, 6H, and 6I.

FIGS. 7A-7D depict that α3β1 integrins mediate the transport of CAKSMGDIVC-displaying phage particles in A549 cells. FIG. 7A: Western blots of the expression of α3 and (31 integrin chain in A549 cells after transduction with shRNA lentivirus particles that targets human ITGA3 and ITGB1 genes. Anti-β actin was used as protein load control. FIG. 7B: Representative images of the cells in culture. Functional assays were performed with A549 cells transduced with shRNA ITGA3 clone #3 and ITGB1 clone #1. FIG. 7C: Anti-α3 integrin chain blocking antibody saturation curve on adherent A549 cells. FIG. 7D: Mathematical modeling of the transport kinetics of phage particles in vitro. Fits of the exponential function (red and blue lines) to mean permeability assay data (markers) for targeted CAKSMGDIVC-displaying phage particles (red) or control insertless phage particles (blue). Pearson correlation coefficient R >0.96 for both show excellent goodness of fit. Data represents mean±SD (n=3). Note: The y-axis is in log10 scale.

FIGS. 8A-8C depict immunohistochemistry and single-cell RNA sequencing of α3β1 integrins transcripts in mouse lung tissue. FIG. 8A: Immunohistochemistry staining of sectioned paraffin-embedded lung tissue with the anti-α3 integrin chain antibody or an isotype control antibody. Representative image of the alveoli or the airways are shown. FIG. 8B: Itga3 and Itgb1 transcripts of mouse α3β1 integrin by scRNA-seq in lung epithelial cell types. FIG. 8C: Predictions of the pharmacokinetic model for peripheral compartment (slowly perfused organs) and MPSsequestration of targeted CAKSMGDIVC-displaying phage particles or control insertless phage particles. Note: The y-axis is in log10 scale.

FIGS. 9A-9F depict expression of α3β1 integrins in lung tissue sections from human patients or non-human primates, and humoral response upon intratracheal administration of CAKSMGDIVC-displaying phage particles. FIG. 9A: Immunofluorescence analysis of the expression of α3β1 integrins (red) in alveolar epithelial cells in lung tissue sections of rhesus monkeys. Alveolar epithelial cells were co-stained with an anti-RAGE (green) antibody and DAPI to stain of cell nuclei (blue). FIG. 9B: Expression of α3β1 integrins in normal human lung tissue sections by immunohistochemistry. FIGS. 9C-9D: Fold change in titer of phage-specific serum IgG (FIG. 9C) or serum IgA (FIG. 9D) relative to the control insertless phage particles. FIGS. 9E-9F: Fold change in titer of CAKSMGDIVC-specific IgG (FIG. 9E) and IgA (FIG. 9F) was calculated dividing the mean of antibody titer from each time point by the mean of antibody titer from the baseline. Scale bar:100 μm.

FIG. 10 depicts that aerosol administration of CAKSMGDIVC-displaying phage particles elicits a robust and specific pulmonary and systemic antibody response in mice. Serum and BALF collected from mice after 14 days of aerosol administration with targeted CAKSMGDIVC-displaying phage particles or control insertless phage particles. Titers of phage-specific IgG, IgM or IgA antibodies from the serum or broncho-alveolar lavage were analyzed by ELISA in 96-well plates coated with 10¹⁰ phage particles per well (***P<0.001). Note: The y-axis is in log2 scale.

### DETAILED DESCRIPTION

The present disclosure relates, in one aspect, to the identification of certain peptides that allow for transport of a solid particle across the air-blood barrier in the lungs. In certain embodiments, the solid particle comprises any sort of solid cargo to which the peptides contemplated in the disclosure can be attached. In other embodiments, the solid particle is further derivatized with a therapeutically useful compound, such as but not limited to an antigen, which can be used to trigger an immune response in the subject to which the compositions of the disclosure are administered. In yet other embodiments, the therapeutically useful compound is displayed on the surface of the solid particle. In yet other embodiments, the therapeutically useful compound is attached to the surface of the solid particle. In yet other embodiments, the therapeutically useful compound is contained within the solid particle.

As described herein, an unbiased combinatorial phage display-based strategy was applied to identify ligand/receptor-mediated pathways for safe and effective transport of particles across the air-blood barrier. The delivery strategy described herein successfully induces systemic effects at a very low risk of lung tissue damage. In certain non-limiting embodiments, the constructs of the disclosure can be used as phage-based vaccines to treat systemic, non-respiratory diseases. An aerosolized phage display ligand library was screened in vivo to isolate targeting motifs capable of crossing intact lung air-blood barriers. The ligand motif CAKSMGDIVC was selected and isolated, and its cognate receptor, the integrin α3β1 on the surface of club cells and alveolar epithelial cells of the lung, was purified. Binding of targeted phage particles displaying the CAKSMGDIVC motif to α3β1 integrin promoted specific phage particle uptake and transport in vitro and in vivo. The findings were validated in a non-human primate model. This non-invasive method of pulmonary delivery of a highly stable antigen carrier (i.e., phage particles) was able to elicit a robust and specific immune response, with unlimited applications for vaccine development. Together, the combinatorial selection system and results discussed herein provide new translational avenues for inhaled therapies and their systemic applications.

To obtain mechanistic insights and explore the diversity of surface receptors implicated in physiological transport of molecules across the air-blood barrier, a combinatorial screening of an aerosolized phage display random peptide library was designed and performed in mice. From the pool of peptide-displaying phage particles recovered from the bloodstream, four dominant ligand peptide candidates mediated phage transport across the pulmonary barrier. Of these selected ligands, the index peptide CAKSMGDIVC showed one of the highest transport efficiencies in vivo, with data suggesting that a specific ligand-receptor interaction likely accounts for targeted pulmonary delivery. The distribution, transport, and clearance of CAKSMGDIVC-displaying phage particles deposited in the airways upon aerosolization were monitored in vivo and ex vivo and indicated that phage transport posed no detectable lung injury without either anatomic or functional pulmonary impairment. These results support the finding that phage particles may be suitable for safe inhaled administration.

To identify the putative receptor(s) for the ligand CAKSMGDIVC peptide, a series of phage binding assays were performed in vitro and in vivo. Specific binding to a human recombinant α3β1 integrin followed by the functional binding and transport of CAKSMGDIVC-displaying phage particles across cell monolayer of an alveolar epithelial surrogate confirmed the ligand-receptor interaction. However, the key evidence that targeted phage particles cross the pulmonary barrier through a ligand-receptor mediated mechanism was unequivocally demonstrated by the specific binding of CAKSMGDIVC-displaying phage particles to α3β1 integrins on the surface of AT1, AT2 and club cells in vivo. α3β1 integrins are expressed at the apical and at the basolateral surfaces of alveolar cells. Without wishing to be limited by any theory, general mechanisms such as transcytosis (active receptor-mediated) or paracellular (passive passage between adjacent cells) transport can be potential facilitators of phage particle traffic into the bloodstream. Thus, ligand-directed delivery through the selective targeting of α3β1 integrins represents a substantial advance over traditional non-targeted aerosol formulations that require penetration enhancers or solubilizing carriers thereby affecting drug stability and dispersion.

To support the translational implications of the ligand peptide-directed pulmonary delivery approach introduced herein, a targeted phage-based vaccination protocol was designed in non-human primates as an aerosolized approach for systemic humoral immunization as a proof-of-concept towards disease vaccination in non-human primates. The selective pulmonary transport of CAKSMGDIVC-displaying phage particles followed by activation of a specific systemic humoral response recapitulated long-held principles of viral vaccinology, and confers superior advances over conventional site-specific vaccination routes.

In certain non-limiting embodiments, phage particles are highly stable under harsh environmental conditions. In other non-limiting embodiments, large-scale production of phage particles is cost-effective (Bao, et al., 2018, Adv Drug Deliv Rev; Barbu, et al., 2016, Phage Therapy in the Era of Synthetic Biology. Cold Spring Harb Perspect Biol 8). In yet other non-limiting embodiments, phage-based vaccines do not induce detectable toxic side-effects (Aghebati-Maleki, et al., 2016, J Biomed Sci 23:66). In yet other non-limiting embodiments, native phage particles have no tropism toward mammalian cells and do not replicate inside eukaryotic cells, and their use is generally considered safe if compared to other classic viral-based vaccination strategies (Barbu, et al., 2016, Cold Spring Harb Perspect Biol 8; Aghebati-Maleki, et al., 2016, J Biomed Sci 23:66). In yet other non-limiting embodiments, unlike conventional peptide vaccines that may often be inactivated due to minimal temperature excursions (˜1° C.), the system introduced herein has no cumbersome and expensive requirements for keeping a so-called “cold-chain” during field applications. In yet other non-limiting embodiments, the ligand-receptor discovery and vaccination properties of the present phage-based system can also be used for the development of targeted pulmonary transgene delivery with libraries of hybrid vectors of adeno-associated virus (AAV) and phage (termed AAVP) (Hajitou, et al., 2006, Cell 125:385-398; Suwan, et al., 2019, PNAS doi: 10.1073/pnas.1906653116). In yet other non-limiting embodiments, phage particles themselves are very strong immunogens, serving as a potent adjuvant to elicit sustained humoral responses (Trepel, et al., 2001, Cancer Res 61:8110-8112).

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, selected materials and methods are described herein. In describing and claiming the present disclosure, the following terminology will be used.

Generally, the nomenclature used herein and the laboratory procedures in cell culture, molecular genetics, pharmacology, protein chemistry, and organic chemistry are those well-known and commonly employed in the art.

Standard techniques are used for biochemical and/or biological manipulations. The techniques and procedures are generally performed according to conventional methods in the art and various general references, which are provided throughout this document.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading can occur within or outside of that particular section. All publications, patents, and patent documents referred to in this document are incorporated by reference herein in their entirety, as though individually incorporated by reference.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Adjuvant” refers to a substance that is capable of potentiating the immunogenicity of an antigen. Adjuvants can be one substance or a mixture of substances and function by acting directly on the immune system or by providing a slow release of an antigen. Examples of adjuvants are aluminum salts, polyanions, bacterial glycopeptides, and slow release agents as Freund's incomplete adjuvant.

The term “ameliorating” or “treating” means that the clinical signs and/or the symptoms associated with a disease are lessened as a result of the actions performed. The signs or symptoms to be monitored will be well known to the skilled clinician.

The term “antibody,” as used herein, refers to an immunoglobulin molecule that specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present disclosure can exist in a variety of forms including, for example, polyclonal antibodies, monoclonal antibodies, Fv, Fab and F(ab)2, as well as single chain antibodies (scFv) and humanized antibodies, and any modifications thereof to enhance or alter effector activity, such as glycosylation or mutations in the Fc domains (Harlow, et al., 1999, In: Using Antibodies: A Laboratory Manual, Cold Spring Harbor Laboratory Press, NY; Harlow, et al., 1989, In: Antibodies: A Laboratory Manual, Cold Spring Harbor, New York; Houston, et al., 1988, Proc. Natl. Acad. Sci. USA 85:5879-5883; Bird, et al., 1988, Science 242:423-426).

The term “antigen” or “Ag” as used herein is defined as a molecule that provokes an immune response. This immune response can involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, by “combination therapy” is meant that a first agent is administered in conjunction with another agent. “In combination with” or “In conjunction with” refers to administration of one treatment modality in addition to another treatment modality. As such, “in combination with” refers to administration of one treatment modality before, during, or after delivery of the other treatment modality to the individual. Such combinations are considered to be part of a single treatment regimen or regime.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “eliciting an immune response” or “immunizing” refer to the process of generating a B cell and/or a T cell response against a heterologous protein.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immunogenicity” as used herein, refers to the innate ability of an antigen or organism to elicit an immune response in an animal when the antigen or organism is administered to the animal. Thus, “enhancing the immunogenicity” refers to increasing the ability of an antigen or organism to elicit an immune response in an animal when the antigen or organism is administered to an animal. The increased ability of an antigen or organism to elicit an immune response can be measured by, among other things, a greater number of antibodies that bind to an antigen or organism, a greater diversity of antibodies to an antigen or organism, a greater number of T-cells specific for an antigen or organism, a greater cytotoxic or helper T-cell response to an antigen or organism, a greater expression of cytokines in response to an antigen, and the like.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE, and subclasses within each class. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but can serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

When “an immunologically effective amount,” “an autoimmune disease-inhibiting effective amount,” or “therapeutic amount” is indicated, the precise amount of the compositions of the present disclosure to be administered can be determined by a physician or researcher with consideration of the disease state.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell of the disclosure. Molecules can be modified in many ways, including chemically, structurally, and functionally. Cells can be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

“Parenteral” administration of an immunogenic composition includes, e.g., subcutaneous (s.c.), intravenous (i.v.), intramuscular (i.m.), or intrasternal injection, or infusion techniques.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. The peptide can be linear or branched, can comprise modified amino acids, and can be interrupted by non-amino acids. The terms also encompass an amino acid polymer modified naturally or by intervention; for example, disulfide bond formation, glycosylation, lipidation, acetylation, phosphorylation, or any other manipulation or modification, such as conjugation with a labeling component. Also included within the definition are, for example, polypeptides and proteins containing one or more analogs of an amino acid (including, for example, unnatural amino acids, and so forth), as well as other modifications known in the art. Polypeptides can occur as single chains or associated chains.

As used herein, the term “pharmaceutical composition” refers to a mixture of at least one compound useful within the disclosure with other chemical components, such as carriers, stabilizers, diluents, adjuvants, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present disclosure within or to the subject such that it can perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds can also be incorporated into the compositions.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

By the term “specifically binds,” as used herein with respect to an antibody, is meant an antibody that recognizes a specific antigen, but does not substantially recognize or bind other molecules in a sample. For example, an antibody that specifically binds to an antigen from one species can also bind to that antigen from one or more species. But, such cross-species reactivity does not itself alter the classification of an antibody as specific. In another example, an antibody that specifically binds to an antigen can also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “subject” is intended to include living organisms in which an immune response can be elicited (e.g., mammals). A “subject” or “patient,” as used therein, can be a human or non-human mammal. Non-human mammals include, for example, non-human primates, and livestock and pets, such as ovine, bovine, porcine, canine, feline and murine mammals. Preferably, the subject is human.

A “target site” or “target sequence” refers to a genomic nucleic acid sequence that defines a portion of a nucleic acid to which a binding molecule can specifically bind under conditions sufficient for binding to occur.

The term “therapeutic” as used herein means a treatment and/or prophylaxis. A therapeutic effect is obtained by suppression, remission, or eradication of a disease state.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds and Compositions

The present disclosure relates, in one aspect, to the identification of certain peptides that allow for transport of a solid particle across the air-blood barrier in the lungs. In certain embodiments, the solid particle is further derivatized with a therapeutically useful compound, such as but not limited to an antigen, which can be used to trigger an immune response in the subject to which the compositions of the disclosure are administered. In yet other embodiments, the therapeutically useful compound is displayed on the surface of the solid particle. In yet other embodiments, the therapeutically useful compound is contained within the solid particle.

In certain embodiments, the transport peptides contemplated within the disclosure include, but are not limited to, CAINSLSRKC (SEQ ID NO:1), CAKSMGDIVC (SEQ ID NO:2), CGRKQVESSC (SEQ ID NO:3), and/or CRGKSAEGTC (SEQ ID NO:4). In certain embodiments, the transport peptides of the disclosure are cyclic, wherein the cysteine at position n and the cysteine at position n+8 form a disulfide bond. In other embodiments, the transport peptides of the disclosure are not cyclic. In yet other embodiments, the transport peptide consists of CAINSLSRKC (SEQ ID NO:1). In yet other embodiments, the transport peptide consists of CAKSMGDIVC (SEQ ID NO:2). In yet other embodiments, the transport peptide consists of CGRKQVESSC (SEQ ID NO:3). In yet other embodiments, the transport peptide consists of CRGKSAEGTC (SEQ ID NO:4). In yet other embodiments, the transport peptide consists essentially of CAINSLSRKC (SEQ ID NO:1). In yet other embodiments, the transport peptide consists essentially of CAKSMGDIVC (SEQ ID NO:2). In yet other embodiments, the transport peptide consists essentially of CGRKQVESSC (SEQ ID NO:3). In yet other embodiments, the transport peptide consists essentially of CRGKSAEGTC (SEQ ID NO:4). In yet other embodiments, the transport peptide comprises CAINSLSRKC (SEQ ID NO:1). In yet other embodiments, the transport peptide comprises CAKSMGDIVC (SEQ ID NO:2). In yet other embodiments, the transport peptide comprises CGRKQVESSC (SEQ ID NO:3). In yet other embodiments, the transport peptide comprises CRGKSAEGTC (SEQ ID NO:4). In yet other embodiments, the transport peptide has at least 70%, 80%, 90%, or 100% homology with the peptide of SEQ ID NO:1, SEQ ID NO:2, SEQ ID NO:3, or SEQ ID NO:4. In yet other embodiments, the transport peptide has at least 70%, 80%, 90%, or 100% identity with the peptide of SEQ ID NO:1, SEQ ID NO:2, SEQ ID NO:3, or SEQ ID NO:4.

In certain embodiments, the transport peptides contemplated within the disclosure include AINSLSRK (SEQ ID NO:5), AKSMGDIV (SEQ ID NO:6), GRKQVESS (SEQ ID NO:7), and/or RGKSAEGT (SEQ ID NO:8). In other embodiments, the transport peptide has at least 70%, 80%, 90%, or 100% homology with the peptide of SEQ ID NO:5, SEQ ID NO:6, SEQ ID NO:7, or SEQ ID NO:8. In yet other embodiments, the transport peptide has at least 70%, 80%, 90%, or 100% identity with the peptide of SEQ ID NO:5, SEQ ID NO:6, SEQ ID NO:7, or SEQ ID NO:8.

In certain embodiments, the transport peptide contemplated in the invention is part of a polypeptide, wherein the N-terminus of the transport peptide is the N-terminus of the polypeptide (i.e., the N-terminus of the transport peptide is not coupled to other amino acids/peptides). In certain embodiments, the transport peptide contemplated in the invention is part of a polypeptide, wherein the N-terminus of the transport peptide is not the N-terminus of the polypeptide. In certain embodiments, the transport peptide contemplated in the invention is part of a polypeptide, wherein the N-terminus of the transport peptide is coupled through an amide bond to the C-terminus of a first amino acid (which is a single amino acid or the C-terminus of a (poly)peptide). In certain embodiments, the first amino acid is aspartate. In certain embodiments, the first amino acid is glutamate. In certain embodiments, the first amino acid is lysine. In certain embodiments, the first amino acid is arginine. In certain embodiments, the first amino acid is histidine. In certain embodiments, the first amino acid is alanine. In certain embodiments, the first amino acid is valine. In certain embodiments, the first amino acid is leucine. In certain embodiments, the first amino acid is isoleucine. In certain embodiments, the first amino acid is proline. In certain embodiments, the first amino acid is phenylalanine. In certain embodiments, the first amino acid is methionine. In certain embodiments, the first amino acid is tryptophan. In certain embodiments, the first amino acid is glycine. In certain embodiments, the first amino acid is asparagine. In certain embodiments, the first amino acid is glutamine. In certain embodiments, the first amino acid is cysteine. In certain embodiments, the first amino acid is serine. In certain embodiments, the first amino acid is threonine. In certain embodiments, the first amino acid is tyrosine. In certain embodiments, the first amino acid is not aspartate. In certain embodiments, the first amino acid is not glutamate. In certain embodiments, the first amino acid is not lysine. In certain embodiments, the first amino acid is not arginine. In certain embodiments, the first amino acid is not histidine. In certain embodiments, the first amino acid is not alanine. In certain embodiments, the first amino acid is not valine. In certain embodiments, the first amino acid is not leucine. In certain embodiments, the first amino acid is not isoleucine. In certain embodiments, the first amino acid is not proline. In certain embodiments, the first amino acid is not phenylalanine. In certain embodiments, the first amino acid is not methionine. In certain embodiments, the first amino acid is not tryptophan. In certain embodiments, the first amino acid is not glycine. In certain embodiments, the first amino acid is not asparagine. In certain embodiments, the first amino acid is not glutamine. In certain embodiments, the first amino acid is not cysteine. In certain embodiments, the first amino acid is not serine. In certain embodiments, the first amino acid is not threonine. In certain embodiments, the first amino acid is not tyrosine.

In certain embodiments, the transport peptide contemplated in the invention is part of a polypeptide, wherein the C-terminus of the transport peptide is the C-terminus of the polypeptide (i.e., the C-terminus of the transport peptide is not coupled to other amino acids/peptides). In certain embodiments, the transport peptide contemplated in the invention is part of a polypeptide, wherein the C-terminus of the transport peptide is not the C-terminus of the polypeptide. In certain embodiments, the transport peptide contemplated in the invention is part of a polypeptide, wherein the C-terminus of the transport peptide is coupled through an amide bond to the N-terminus of a second amino acid (which is a single amino acid or the N-terminus of a (poly)peptide). In certain embodiments, the second amino acid is aspartate. In certain embodiments, the second amino acid is glutamate. In certain embodiments, the second amino acid is lysine. In certain embodiments, the second amino acid is arginine. In certain embodiments, the second amino acid is histidine. In certain embodiments, the second amino acid is alanine. In certain embodiments, the second amino acid is valine. In certain embodiments, the second amino acid is leucine. In certain embodiments, the second amino acid is isoleucine. In certain embodiments, the second amino acid is proline. In certain embodiments, the second amino acid is phenylalanine. In certain embodiments, the second amino acid is methionine. In certain embodiments, the second amino acid is tryptophan. In certain embodiments, the second amino acid is glycine. In certain embodiments, the second amino acid is asparagine. In certain embodiments, the second amino acid is glutamine. In certain embodiments, the second amino acid is cysteine. In certain embodiments, the second amino acid is serine. In certain embodiments, the second amino acid is threonine. In certain embodiments, the second amino acid is tyrosine. In certain embodiments, the second amino acid is not aspartate. In certain embodiments, the second amino acid is not glutamate. In certain embodiments, the second amino acid is not lysine. In certain embodiments, the second amino acid is not arginine. In certain embodiments, the second amino acid is not histidine. In certain embodiments, the second amino acid is not alanine. In certain embodiments, the second amino acid is not valine. In certain embodiments, the second amino acid is not leucine. In certain embodiments, the second amino acid is not isoleucine. In certain embodiments, the second amino acid is not proline. In certain embodiments, the second amino acid is not phenylalanine. In certain embodiments, the second amino acid is not methionine. In certain embodiments, the second amino acid is not tryptophan. In certain embodiments, the second amino acid is not glycine. In certain embodiments, the second amino acid is not asparagine. In certain embodiments, the second amino acid is not glutamine. In certain embodiments, the second amino acid is not cysteine. In certain embodiments, the second amino acid is not serine. In certain embodiments, the second amino acid is not threonine. In certain embodiments, the second amino acid is not tyrosine.

Conservative amino acid replacements, i.e., replacements of one amino acid with another which has a related side chain, are also contemplated herein. Genetically-encoded amino acids are generally divided into four families: (1) acidic, i.e., aspartate, glutamate; (2) basic, i.e., lysine, arginine, histidine; (3) non polar, i.e., alanine, valine, leucine, isoleucine, proline, phenylalanine, methionine, tryptophan; and (4) uncharged polar, i.e., glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides can have one or more (e.g., 1, 2, 3, and so forth) single amino acid deletions relative to the exemplified sequences. The polypeptides can also include one or more (e.g., 1, 2, 3, and so forth) insertions relative to the exemplified sequences.

The disclosure further contemplates any nucleic acid sequences encoding any of the transport peptides of the disclosure, as well as any vectors comprising any nucleic acid sequences encoding any of the transport peptides of the disclosure, as well as any cells comprising any vector comprising any nucleic acid sequences encoding any of the transport peptides of the disclosure. The disclosure further contemplates nucleic acid sequences that have about 85%, 86%, 87%, 88%, 89%, 90%, 91%, 92%, 93%, 94%, 95%, 96%, 97%, 98%, or 99% sequence identity to the nucleic acid sequences provided herein.

In certain embodiments, at least one residue within the transport peptide, and/or at the carboxy-terminus of the transport peptide, and/or at the amino-terminus of the transport peptide is methylated, amidated, acylated (such as, but not limited to, acetylated), and/or substituted with any other chemical group without adversely affecting activity of the transport peptide within the compositions and/or methods of the disclosure. In other embodiments, the N-terminus of the transport peptide is acylated, such as but not limited to acetylated. In other embodiments, the C-terminus of the transport peptide is amidated.

In certain embodiments, the disclosure provides a solid particle, wherein the transport peptide is displayed on the surface of the solid particle. In other embodiments, the transport peptide is attached to the surface of the solid particle. In yet other embodiments, the transport peptide is covalently attached to the surface of the solid particle. In yet other embodiments, the solid particle is selected from the group consisting of a phage, engineered cell, tissue fragment, nanoparticle, vesicle, dendrimer, virus-like particle (VLP), adenovirus, adeno-associated virus (AAV), adeno-associated virus phage (termed AAVP), and any combinations thereof. In some instances, a nanoparticle has a diameter on the nanometer scale, and can vary from about 1 nm in diameter to about 1,000 nm in diameter. In some instances, a phage has a diameter that is lower than about 10 nm, such as but not limited to about 1 nm, 2 nm, 3 nm, 4 nm, 5 nm, 6 nm, 7 nm, 8 nm, 9 nm, or 10 nm. In some instances, a phage has a length that is lower than 1,000 nm, such as but not limited to about 100 nm, 200 nm, 300 nm, 400 nm, 500 nm, 600 nm, 700 nm, 800 nm, 900 nm, or 1,000 nm. In yet another embodiments, the composition further comprises an agent selected from the group consisting of a therapeutic agent, biologically active molecule, imaging agent, radioactive agent, salt, peptide, protein, lipid, nucleic acid, gas, and any combinations thereof, wherein the agent is attached to and/or contained within the solid particle. In certain embodiments, the transport peptide is attached to and/or displayed on the whole surface of the solid particle. In other embodiments, the transport peptide is attached to and/or displayed on at least a fraction of the surface of the solid particle. The solid particles can be prepared using methods known to those skilled in the art or purchased from commercial sources.

In some embodiments, the disclosure provides a vaccine composition comprising a transport peptide described elsewhere herein. In some embodiments, the vaccine composition is a live attenuated vaccine, an inactivated vaccine, a subunit, recombinant, polysaccharide, or conjugated vaccine, and/or a toxin vaccine. In some embodiments, the vaccine composition comprises a DNA vaccine, RNA vaccine, replicating viral vector vaccine, non-replicating viral vector vaccine, inactivated viral vector vaccine, and/or virus-like particle vaccine that is known to be useful for nasal, buccal, inhalational, intratracheal, intrapulmonary, and/or intrabronchial delivery. In some embodiments, the vaccine composition comprises an adjuvant. Exemplary adjuvants are described elsewhere herein.

The transport peptides of the disclosure can be synthesized using chemical and biochemical methods known to those skilled in the art of chemical synthesis or peptide synthesis. The transport peptides can be attached to the surface of a solid particle using any method known to those skilled in the art. In certain embodiments, the transport peptides can be attached to the surface of a solid particle via a covalent bond. In a non-limiting example, a free amino group in the transport peptide can be attached to free carboxylate groups on the surface of a solid particle via covalent amide bonds. In a non-limiting example, a free carboxylic acid group in the transport peptide can be attached to free amino groups on the surface of a solid particle via covalent amide bonds. In other embodiments, the transport peptide can be attached to the surface of a solid particle via a non-covalent bond.

In certain embodiments, the solid particle is a bacteriophage, such as but not limited to a filamentous phage. The filamentous bacteriophage can include, but are not limited to fd, fl, or M13 bacteriophage. In certain embodiments, the bacteriophage is a fd bacteriophage. In other embodiments, the bacteriophage is T4, T7, or λ phage. In some embodiments, the filamentous phage has a diameter of equal to or less than about 10 nm. In some embodiments, the filamentous phage has a diameter of equal to or less than about 9 nm. In some embodiments, the filamentous phage has a diameter equal to or less than about 8 nm. In some embodiments, the filamentous phage has a diameter equal to or less than about 7 nm. In some embodiments, the filamentous phage has a diameter equal to or less than about 6 nm. In some embodiments, the filamentous phage has a diameter equal to or less than about 5 nm. In some embodiments, the filamentous phage has a diameter equal to or less than about 4 nm. In some embodiments, the filamentous phage has a diameter equal to or less than about 3 nm. In some embodiments, the filamentous phage has a diameter equal to or less than about 2 nm. In some embodiments, the filamentous phage has a diameter equal to or less than about 1 nm. In some embodiments, the filamentous phage has a diameter equal to or greater than about 9 nm. In some embodiments, the filamentous phage has a diameter equal to or greater than about 8 nm. In some embodiments, the filamentous phage has a diameter equal to or greater than about 7 nm. In some embodiments, the filamentous phage has a diameter equal to or greater than about 6 nm. In some embodiments, the filamentous phage has a diameter equal to or greater than about 5 nm. In some embodiments, the filamentous phage has a diameter equal to or greater than about 4 nm. In some embodiments, the filamentous phage has a diameter equal to or greater than about 3 nm. In some embodiments, the filamentous phage has a diameter equal to or greater than about 2 nm. In some embodiments, the filamentous phage has a diameter equal to or greater than about 1 nm.

Typically, filamentous phage (M13, fd, fl) have a filamentous capsid with a circular ssDNA molecule. The genome typically contains 10 genes but none for a lysis protein. Virions are enveloped. The filamentous phage typically infect only E. coli cells carrying the F plasmid since the phage must adsorb to the F pilus to gain entry to the cells. Their life cycle involves a dsDNA intermediate replicative form within the cell, which is converted to a ssDNA molecule prior to encapsidation. Phages provide an easy means to prepare ssDNA for DNA sequencing. The best known example is bacteriophage M13, which has been adapted for use as a cloning and sequencing vector. The wild-type M13 genome is 6,407 bp in length. Other relatives of M13 are fd and fl. The phage modified cloning vector fUSE5 has approximately 9,200 pb in length.

Various methods of phage display and methods for producing diverse populations of peptides are well known in the art. For example, U.S. Pat. Nos. 5,223,409; 5,622,699; 5,866,363; and 6,068,829; and JP Patent No. 4875497 B2; each of which is incorporated herein by reference, describe methods for preparing a phage library. The phage display technique involves genetically manipulating bacteriophage so that small peptides can be expressed on their surface [Smith, 1985, Science 228(4705):1315-1317]. In this technique, a gene encoding a protein of interest is inserted into a phage coat protein gene, causing the phage to “display” the protein on its outside while containing the gene for the protein on its inside, resulting in a connection between genotype and phenotype. In the case of M13 filamentous phage display, the DNA encoding the protein or peptide of interest is ligated into the pIII or pVIII gene, encoding either the minor or major coat protein, respectively. Multiple cloning sites are sometimes used to ensure that the fragments are inserted in all three possible reading frames so that the cDNA fragment is translated in the proper frame. The phage gene and insert DNA hybrid is then inserted (a process known as “transduction”) into E. coli bacterial cells such as TG1, SS320, ER2738, or XL1-Blue E. coli. If a “phagemid” vector is used, phage particles are not released from the E. coli cells until they are infected with helper phage, which enables packaging of the phage DNA and assembly of the mature virions with the relevant protein fragment as part of their outer coat on either the minor (pIII) or major (pVIII) coat protein.

It should be noted that phage display methods can be applied not only to the transport peptides but also to any peptide and/or protein that should be displayed on the surface of the phage (such as, but not limited to, a biologically active peptide and/or antigen).

Peptides and proteins contemplated in the disclosure can be prepared in several known ways, e.g., by chemical synthesis (in whole or in part), by digesting longer polypeptides using proteases, by translation from RNA, by purification from cell culture (e.g., from recombinant expression), from the organism itself (e.g., after bacterial culture, or direct from patients), and so forth. Processes for producing proteins of the disclosure are known to those skilled in the art. For example, protein production can comprise the step of culturing a host cell of the disclosure under conditions which induce protein expression.

A non-limiting method for production of peptides <40 amino acids long involves in vitro chemical synthesis (Raddrizzani, et al., 2000, Briefs in Bioinformatics 14(2):121-130; Fields, et al., 1997, Principles of Peptide Synthesis. ISBN: 0387564314). Solid-phase peptide synthesis is available, such as methods based on tPoc or Fmoc chemistry (Chan, et al., 2000, Fmoc solid phase peptide synthesis. ISBN:0849368413). Enzymatic synthesis can also be used in part or in full. As an alternative to chemical synthesis, biological synthesis can be used, e.g., the polypeptides can be produced by translation. This can be carried out in vitro or in vivo. Biological methods are in general restricted to the production of polypeptides based on L-amino acids, but manipulation of translation machinery (e.g., of aminoacyl tRNA molecules) can be used to allow the introduction of D-amino acids (or of other non-natural amino acids, such as iodotyrosine or methylphenylalanine, azidohomoalanine, and so forth) (Ibba, 1996, Biotechnology and Genetic Engineering Review 13:197-216). Where D-amino acids are included, however, it is possible to use chemical synthesis. Proteins of the disclosure can have covalent modifications at the C-terminus and/or N-terminus.

Proteins useful within the disclosure can take various forms (e.g., native, fusions, glycosylated, non-glycosylated, lipidated, non-lipidated, phosphorylated, non-phosphorylated, myristoylated, non-myristoylated, monomeric, multimeric, particulate, denatured, and so forth). Proteins of the disclosure can be provided in purified or substantially purified form, i.e., substantially free from other polypeptides (e.g., free from naturally occurring polypeptides), and are generally at least about 50% pure (by weight), and usually at least about 90% pure, i.e., less than about 50%, and more preferably less than about 10% (e.g. 5%) of a composition, is made up of other expressed proteins.

Polypeptides of the disclosure can comprise a detectable label (e.g., a radioactive or fluorescent label, or a biotin label). Proteins of the disclosure can be naturally or non-naturally glycosylated (i.e., the polypeptide has a glycosylation pattern that differs from the glycosylation pattern found in the corresponding naturally occurring polypeptide).

Various tests can be used to assess the in vivo immunogenicity of proteins of the disclosure. For example, polypeptides can be expressed recombinantly and used to screen patient sera by immunoblot. A positive reaction between the polypeptide and patient serum indicates that the patient has previously mounted an immune response, specifically an antibody response, to the protein in question, i.e., the protein is an immunogen. This method can also be used to identify immunodominant proteins.

### Methods

In one aspect, the disclosure provides a method of promoting or increasing transport of a solid particle across the air-blood barrier in the lung of a subject. In certain embodiments, the method comprises administering to the subject the solid particle to which surface a transport peptide of the disclosure is attached. In other embodiments, the administration is through a route comprising nasal, buccal, inhalational, intratracheal, intrapulmonary, or intrabronchial.

In one aspect, the disclosure provides a method of promoting systemic circulation of a solid particle in a subject. In certain embodiments, the method comprises administering to the subject the solid particle to which surface a transport peptide of the disclosure is attached. In other embodiments, the administration is through a route comprising nasal, buccal, inhalational, intratracheal, intrapulmonary, or intrabronchial.

In one aspect, the disclosure provides a method of immunizing a subject against a disease or disorder. In certain embodiments, the method comprises administering to the subject the solid particle to which surface a transport peptide of the disclosure is attached, wherein the surface of the solid particle is further derivatized with an antigen that promotes immune response to the disease or disorder. In other embodiments, the administration is through a route comprising nasal, buccal, inhalational, intratracheal, intrapulmonary, or intrabronchial.

In another aspect, the disclosure provides a method of vaccinating a subject against a disease or disorder. In certain embodiments, the method comprises administering to the subject a vaccine comprising the solid particle to which surface a transport peptide of the disclosure is attached, wherein the surface of the solid particle is further derivatized with an antigen that promotes immune response to the disease or disorder. In other embodiments, the administration is through a route comprising nasal, buccal, inhalational, intratracheal, intrapulmonary, or intrabronchial.

In one aspect, the disclosure provides a method of treating and/or preventing a disease or disorder in a subject. In certain embodiments, the method comprises administering to the subject the solid particle to which surface a transport peptide of the disclosure is attached, wherein the surface of the solid particle is further derivatized with an antigen that promotes immune response to the disease or disorder. In other embodiments, the administration is through a route comprising nasal, buccal, inhalational, intratracheal, intrapulmonary, or intrabronchial.

In one aspect, the disclosure provides a method of treating a subject at risk of developing a disease or disorder. In certain embodiments, the method comprises administering to the subject the solid particle to which surface a transport peptide of the disclosure is attached, wherein the surface of the solid particle is further derivatized with an antigen that promotes immune response to the disease or disorder. In other embodiments, the administration is through a route comprising nasal, buccal, inhalational, intratracheal, intrapulmonary, or intrabronchial.

Compositions of the present disclosure can be administered in a manner appropriate to the disease or disorder to be treated (or prevented). The quantity and frequency of administration will be determined by such factors as the condition of the patient, and the type and severity of the patient's disease, although appropriate dosages and schedules can be determined by clinical trials.

Compositions of the disclosure can generally be administered directly to a patient. Direct delivery can be accomplished by parenteral injection (e.g., subcutaneously, intraperitoneally, intravenously, intramuscularly, or to the interstitial space of a tissue), or by rectal, oral, vaginal, topical, transdermal, intranasal, sublingual, ocular, aural, pulmonary or other mucosal administration. In certain embodiments, the administration is through a route comprising nasal, buccal, inhalational, intratracheal, intrapulmonary, or intrabronchial.

Dosage treatment can be a single dose schedule or a multiple dose schedule. For example, multiple doses can be used in a primary immunization schedule and/or in a booster immunization schedule. A primary dose schedule can be followed by a booster dose schedule. Suitable timing between priming doses (e.g., between 4-16 weeks), and between priming and boosting, can be routinely determined.

Solid forms suitable for solution in, or suspension in, liquid vehicles prior to injection can also be prepared (e.g., a lyophilized composition). The composition can be prepared for pulmonary administration, e.g., as an inhaler, using a fine powder or a spray. The composition can be prepared for nasal, aural or ocular administration, e.g., as spray, drops, gel or powder, e.g., Almeida, et al., 1996, J. Drug Targeting 3:455-467.

Antigens in the composition can typically be present at a concentration of at least 1 μg/ml each. In general, the concentration of any given antigen will be sufficient to elicit an immune response against that antigen.

Pharmaceutical compositions

Certain embodiments of the disclosure are directed to prophylactically treating an individual in need thereof. As used herein, the term “prophylactic treatment” includes, but is not limited to, the administration of an antigen to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder.

Certain embodiments of the disclosure are directed to therapeutically treating an individual in need thereof. As used herein, the term “therapeutically” includes, but is not limited to, the administration of an antigen to a subject who displays symptoms or signs of pathology, disease, or disorder, in which treatment is administered to the subject for the purpose of diminishing or eliminating those signs or symptoms of pathology, disease, or disorder.

Embodiments of the present disclosure are directed to compositions and methods for enhancing the immune response of a subject to one or more antigens. As used herein, the terms “subject” and “host” are intended to include living organisms such as mammals. Examples of subjects or hosts include, but are not limited to, horses, cows, sheep, pigs, goats, dogs, cats, rabbits, guinea pigs, rats, mice, gerbils, non-human primates, humans and the like, non-mammals, including, e.g., non-mammalian vertebrates, such as birds (e.g., chickens or ducks) fish or frogs (e.g., Xenopus), and a non-mammalian invertebrates, as well as transgenic species thereof. Preferably, the subject is a human.

Compositions of the disclosure can include one or more pharmaceutically or physiologically acceptable carriers. A pharmaceutically acceptable carrier is a compound that does not itself induce harmful effects to the individual receiving the composition. Suitable carriers are typically large, slowly metabolized macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The vaccines can also contain diluents, such as water, saline, glycerol, and so forth. Additionally, auxiliary substances, such as wetting or emulsifying agents, pH buffering substances, and the like, can be present. Sterile pyrogen-free, phosphate-buffered, physiologic saline is a typical carrier.

Compositions of the disclosure can include an antimicrobial, particularly if packages in a multiple-dose format. Compositions of the disclosure can comprise a detergent, e.g., a Tween (polysorbate), such as Tween 80. Detergents are generally present at low levels, e.g., <0.1%.

Compositions of the disclosure can include sodium salts (e.g., sodium chloride) to give tonicity. A concentration of 10±2 mg/ml sodium chloride is typical. Compositions of the disclosure can generally include a buffer. A phosphate buffer is typical. Compositions of the disclosure can comprise a sugar alcohol (e.g., mannitol) or a disaccharide (e.g., sucrose or trehalose), e.g., at around 15-30 mg/ml (e.g., 25 mg/ml), particularly if they are to be lyophilized or if they include material which has been reconstituted from lyophilized material. The pH of a composition for lyophilization can be adjusted to around 6.1 prior to injection.

Compositions of the disclosure can include an immunogenic adjuvant. An adjuvant is a pharmacological or immunological agent that modifies the effect of other agents. Adjuvants can be added to a vaccine to boost the immune response to produce more antibodies and longer-lasting immunity, thus minimizing the dose of antigen needed. Adjuvants can also be used to enhance the efficacy of a vaccine by helping to modify the immune response to particular types of immune system cells: for example, by activating T cells instead of antibody-secreting B cells depending on the purpose of the vaccine. Immunogenic adjuvants include but are not limited to alum, MF59, AS03, Virosome, AS04, aluminum hydroxide, and paraffin oil.

Mineral containing compositions suitable for use as adjuvants in the disclosure include mineral salts, such as aluminum salts and calcium salts. The disclosure includes mineral salts such as hydroxides (e.g., oxyhydroxides), phosphates (e.g., hydroxyphosphates, orthophosphates), sulphates, and so forth, or mixtures of different mineral compounds, with the compounds taking any suitable form (e.g., gel, crystalline, amorphous, and so forth), and with adsorption being preferred. The mineral containing compositions can also be formulated as a particle of metal salt.

Aluminum phosphates are useful, particularly in compositions which include an oligosaccharide antigen, and a typical adjuvant is amorphous aluminum hydroxyphosphate with PO/A1 molar ratio between 0.84 and 0.92, included at 0.6 mg Al/ml. Adsorption with a low dose of aluminum phosphate can be used, e.g., between 50 and 100 μg per conjugate per dose.

Oil emulsion compositions suitable for use as adjuvants in the disclosure include squalene-water emulsions, such as MF59 (5% Squalene, 0.5% Tween 80, 0.5% Span 85, formulated into Submicron particles using a microfluidizer). Complete Freund's adjuvant (CFA) and incomplete Freund's adjuvant (IFA) can also be used.

Saponin compositions can also be used as adjuvants in the disclosure. Saponins are a heterologous group of sterol glycosides and triterpenoid glycosides that are found in the bark, leaves, stems, roots and even flowers of a wide range of plant species. Saponin from the bark of the Ouillaia saponaria Molina tree have been widely studied as adjuvants. Saponin can also be commercially obtained from Smilax ornata (sarsaprilla), Gypsophilla paniculata (brides Veil), and Chlorogalum pomeridianum (soap root). Saponin adjuvant formulations include purified formulations, such as QS21, as well as lipid formulations, such as ISCOMs. QS21 is marketed as Stimulon™.

Additional adjuvants suitable for use in the disclosure include bacterial or microbial derivatives such as non-toxic derivatives of enterobacterial lipopolysaccharide (LPS), Lipid A derivatives, immunostimulatory oligonucleotides and ADP ribosylating toxins and detoxified derivatives thereof. Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A “small particle” form of 3 De-O-acylated monophosphoryl lipid A is also available. Such “small particles” of 3d MPL are small enough to be sterile filtered through a 0.22 um membrane. Other non-toxic LPS derivatives include monophosphoryl lipid A mimics, such as aminoalkyl glucosaminide phosphate derivatives, e.g., RC-52950,51. Lipid A derivatives include derivatives of lipid A from Escherichia coli such as OM-174.

Immunostimulatory oligonucleotides suitable for use as adjuvants in the disclosure include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory. The CpG's can include nucleotide modifications/analogs such as phosphorothioate modifications and can be double-stranded or single-stranded. The CpG sequence can be directed to TLR9, such as the motif GTCGTT or TTCGTT. The CpG sequence can be specific for inducing a Th1 immune response, such as a CpG-A ODN, or it can be more specific for inducing a B cell response, such a CpG-BODN. Preferably, the CpG is a CpG-A ODN.

Preferably, the CpG oligonucleotide is constructed so that the 5′ end is accessible for receptor recognition. Optionally, two CpG oligonucleotide sequences can be attached at their 3′ ends to form “immunomers.” Bacterial ADP-ribosylating toxins and detoxified derivatives thereof can be used as adjuvants in the disclosure. Preferably, the protein is derived from E.coli (E.coli heat labile enterotoxin“LT”), cholera (“CT), or pertussis (”PT). The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. ADP-ribosylating toxins and detoxified derivatives thereof, particularly LT-K, can be used. Numerical reference for amino acid substitutions is preferably based on the alignments of the A and B subunits of ADP-ribosylating toxins.

### Administration/Dosage/Formulations

The regimen of administration can affect what constitutes an effective amount. The therapeutic formulations can be administered to the subject either prior to or after the onset of a disease or disorder contemplated in the disclosure. Further, several divided dosages, as well as staggered dosages can be administered daily or sequentially, or the dose can be continuously infused, or can be a bolus injection. Further, the dosages of the therapeutic formulations can be proportionally increased or decreased as indicated by the exigencies of the therapeutic or prophylactic situation.

Administration of the compositions of the present disclosure to a patient, such as a mammal, such as a human, can be carried out using known procedures, at dosages and for periods of time effective to treat a disease or disorder contemplated in the disclosure. An effective amount of the therapeutic compound necessary to achieve a therapeutic effect can vary according to factors such as the state of the disease or disorder in the patient; the age, sex, and weight of the patient; and the ability of the therapeutic compound to treat a disease or disorder contemplated in the disclosure. Dosage regimens can be adjusted to provide the optimum therapeutic response. For example, several divided doses can be administered daily or the dose can be proportionally reduced as indicated by the exigencies of the therapeutic situation. A non-limiting example of an effective dose range for a therapeutic compound of the disclosure is from about 1 and 5,000 mg/kg of body weight/per day. One of ordinary skill in the art would be able to study the relevant factors and make the determination regarding the effective amount of the therapeutic compound without undue experimentation.

In certain embodiments, the effective dose range is measured in units known to a person of skill in the art to be suitable for the description of phage doses. In some embodiments, the effective dose range for a vaccine or therapeutic compound of the disclosure is measured by transducing units (TU)/kg/day or particles/kg/day. In some embodiments, the dosage provided to a patient is between about 10⁶-10¹² TU/kg/day. In some embodiments, the effective dose range is measured by plaque forming units (PFU), colony forming units (CFU), 50% tissue culture infectious dose (TCID50), plaque reduction neutralization test (PRNT), and combinations thereof.

Actual dosage levels of the active ingredients in the pharmaceutical compositions of this disclosure can be varied so as to obtain an amount of the active ingredient that is effective to achieve the desired therapeutic response for a particular patient, composition, and mode of administration, without being toxic to the patient.

The therapeutically effective amount or dose of a compound of the present disclosure depends on the age, sex and weight of the patient, the current medical condition of the patient and the progression of a disease or disorder contemplated in the disclosure.

A medical doctor, e.g., physician or veterinarian, having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In certain embodiments, the compositions of the disclosure are administered to the patient in dosages that range from one to five times per day or more. In other embodiments, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

It is understood that the amount of compound dosed per day can be administered, in non-limiting examples, every day, every other day, every 2 days, every 3 days, every 4 days, every 5 days, every week, every two weeks, every three weeks, every four weeks, or every month. For example, with every other day administration, a 5 mg per day dose can be initiated on Monday with a first subsequent 5 mg per day dose administered on Wednesday, a second subsequent 5 mg per day dose administered on Friday, and so on. As a second example, with every four week administration for immunization purposes, each dose can be administered every 28 days. In certain embodiments wherein the disclosed formulations or compositions are administered for immunization purposes every 28 days, serum is collected every 14 days.

In the case wherein the patient's status does improve, upon the doctor's discretion the administration of the inhibitor of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In certain embodiments, patients require intermittent treatment on a long-term basis upon any recurrence of symptoms and/or infection.

The compounds for use in the method of the disclosure can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies in certain embodiments within a range of circulating concentrations that include the ED₅₀ with minimal toxicity. The dosage optionally varies within this range depending upon the dosage form employed and the route of administration utilized.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier can be a solvent or dispersion medium containing, for example, saline, buffered saline, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it is advisable to include isotonic agents, for example, sugars, sodium chloride, or polyalcohols such as mannitol and sorbitol, in the composition.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder contemplated in the disclosure.

Formulations can be employed in admixtures with conventional excipients, i.e., pharmaceutically acceptable organic or inorganic carrier substances suitable for any suitable mode of administration, known to the art. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., analgesic agents.

Routes of administration of any of the compositions of the disclosure include oral, nasal, pulmonary, rectal, intravaginal, parenteral, buccal, sublingual, or topical. The compounds for use in the disclosure can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In certain embodiments, routes of administration of any of the compositions of the disclosure include nasal, buccal, inhalational, intratracheal, intrapulmonary, and intrabronchial.

Suitable compositions and dosage forms include, for example, dispersions, suspensions, solutions, syrups, granules, beads, powders, pellets, liquid sprays for nasal or oral administration, dry powder or aerosolized formulations for inhalation, and the like. It should be understood that the formulations and compositions that would be useful in the present disclosure are not limited to the particular formulations and compositions that are described herein.

Powdered and granular formulations of a pharmaceutical preparation of the disclosure can be prepared using known methods. Such formulations can be administered directly to a subject, used, for example, to form a material that is suitable to administration to a subject. Each of these formulations can further comprise one or more of dispersing or wetting agent, a suspending agent, and a preservative. Additional excipients, such as fillers and sweetening, flavoring, or coloring agents, can also be included in these formulations.

### Oral Administration

For oral application, particularly suitable are tablets, dragees, liquids, drops, suppositories, or capsules, caplets and gelcaps. The compositions intended for oral use can be prepared according to any method known in the art and such compositions can contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets can be uncoated or they can be coated by known techniques for elegance or to delay the release of the active ingredients. Formulations for oral use can also be presented as hard gelatin capsules wherein the active ingredient is mixed with an inert diluent.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

### Buccal, Pulmonary, Inhalational, Intranasal Administration, and So Forth

A pharmaceutical composition of the disclosure can be prepared, packaged, or sold in a formulation suitable for pulmonary administration via the buccal cavity. Such a formulation can be a liquid or dry/powder formulation comprising one or more targeting peptides of the disclosure. In some embodiments, the formulation comprises an active ingredient described elsewhere herein. In some embodiments, the particles of the dry/powder formulation have a diameter in the range from about 0.5 to about 7 micrometers, and in certain embodiments from about 1 to about 6 micrometers. Such compositions are conveniently in the form of dry powders for administration using a device comprising a dry powder reservoir to which a stream of propellant can be directed to disperse the powder or using a self-propelling solvent/powder-dispensing container such as a device comprising the active ingredient dissolved or suspended in a low-boiling propellant in a sealed container. In certain embodiments, such powders comprise particles wherein at least 98% of the particles by weight have a diameter greater than 0.5 micrometers and at least 95% of the particles by number have a diameter less than 7 micrometers. In certain embodiments, at least 95% of the particles by weight have a diameter greater than 1 micrometer and at least 90% of the particles by number have a diameter less than 6 micrometers. Dry powder compositions can include a solid fine powder diluent such as sugar and are conveniently provided in a unit dose form. See also EP Patents No. EP 02 12 753B1 and No. 1 370 318B1.

Low boiling propellants generally include liquid propellants having a boiling point of below 65° F. at atmospheric pressure. Generally the propellant can constitute 50 to 99.9% (w/w) of the composition, and the active ingredient can constitute 0.1 to 20% (w/w) of the composition. The propellant can further comprise additional ingredients such as a liquid non-ionic or solid anionic surfactant or a solid diluent (in certain embodiments having a particle size of the same order as particles comprising the active ingredient).

Pharmaceutical compositions of the disclosure formulated for pulmonary delivery can also provide the active ingredient in the form of droplets of a solution or suspension. Such formulations can be prepared, packaged, or sold as aqueous or dilute alcoholic solutions or suspensions, optionally sterile, comprising the active ingredient, and can conveniently be administered using any nebulization or atomization device. Such formulations can further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration in certain embodiments have an average diameter in the range from about 0.1 to about 200 micrometers.

The pharmaceutical composition of the disclosure can be delivered using an inhalator such as those recited in U.S. Pat. No. 8,333,192 B2, which is incorporated herein by reference in its entirety.

The formulations described herein as being useful for pulmonary delivery are also useful for intranasal delivery of a pharmaceutical composition of the disclosure.

Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares. Formulations suitable for nasal administration may, for example, comprise from about as little as 0.1% (w/w) and as much as 100% (w/w) of the active ingredient, and can further comprise one or more of the additional ingredients described herein.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. Patent Applications Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time can be as long as a month or more and should be a release which is longer that the same amount of agent administered in bolus form.

For sustained release, the compounds can be formulated with a suitable polymer or hydrophobic material that provides sustained release properties to the compounds. As such, the compounds for use the method of the disclosure can be administered in the form of microparticles, for example, by injection or in the form of wafers or discs by implantation.

In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, 4th edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the disclosure, and, as such, can be considered in making and practicing the disclosure.

It should be understood that the method and compositions that would be useful in the present disclosure are not limited to the particular formulations set forth in the examples. The following examples are put forth so as to provide those of ordinary skill in the art with a complete disclosure and description of how to make and use the cells, expansion and culture methods, and therapeutic methods of the disclosure, and are not intended to limit the scope of what the inventors regard as their disclosure.

### EXPERIMENTAL EXAMPLES

The disclosure is now described with reference to the following Examples. These Examples are provided for the purpose of illustration only, and the disclosure is not limited to these Examples, but rather encompasses all variations that are evident as a result of the teachings provided herein.

### Materials and Methods

### Animals:

BALB/c mice were purchased from The Jackson Laboratory (Sacramento, CA). The Institutional Care and Use Committees (IACUCs) of the University of Texas M.D. Anderson Cancer Center (UTMDACC), the University of New Mexico Health Sciences Center, and Rutgers Cancer Institute of New Jersey approved all animal experiments. Adult rhesus macaques used in the vaccination study were housed at Michale E. Keeling Center for Comparative Medicine and Research, an Association for Assessment and Accreditation of Laboratory Animal Care (AAALAC)-accredited veterinary facility at the University of Texas M.D. Anderson Cancer Center (UTMDACC). For aerosol administration, trained mice and primate handlers followed the National Research Council's Guide for the Care and Use of Laboratory Animals.

### Tissue Culture:

Human alveolar epithelial adenocarcinoma A549 cells were purchased from American Type Culture Collection (ATCC) and maintained in Dulbecco's Modified Eagle's Minimum Essential Medium (DMEM) supplemented with 10% fetal bovine serum (FBS), vitamins, non-essential amino acids, penicillin/streptomycin, and L-glutamine (Gibco) at 37° C. in a 5% CO₂ humidified incubator.

### In Vivo Selection of Aerosolized Phage Display Library:

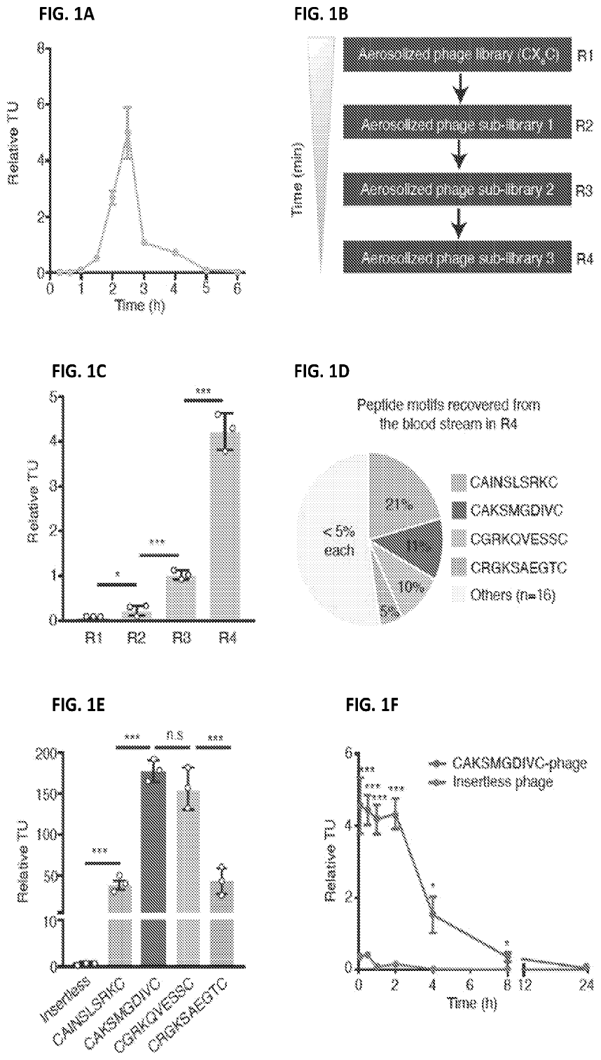

To identify ligand peptide sequences that mediate phage particle delivery through the pulmonary barrier and into the blood stream, a random phage peptide library displaying the insert CX8C (where X is any amino acid residue and C is a cysteine residue) was used for the in vivo screening. Six to eight weeks old BALB/c females were used. Phage input in mice was 10⁹ TU per mouse. The animals were aerosolized via the intratracheal route with 50 μL of phosphate-buffered saline (PBS) containing the phage library with a MICRO SPRAYER® Aerosolizer coupled to a high-pressure syringe (Penn-Century) and a small animal laryngoscope (Penn-Century). The devices were used to administer air-free liquid aerosol directly into the trachea of animals deeply anesthetized with isoflurane (1%). The four rounds of selection were performed as described. In round 1 (R1), animals received 10⁹ TU of the CX8C library by aerosolization (see scheme in FIG. 1B). After 1 h, phage particles were recovered from the bloodstream, amplified and pooled as R1. In round 2 (R2), R1-pooled phage were administered and recovered 30 min post-aerosolization. The subsequent R2 was amplified and pooled for administration in round 3 (R3). After 10 min, the R3-pooled phage was recovered and processed for aerosolization in the final round 4 (R4). After 5 min, phage particles were recovered from the bloodstream, amplified and sequenced. Purification of phage particles and DNA sequencing of phage were performed as described (Arap, et al., 1998, Science 279:377-380).

### Antibodies and Reagents:

Anti-mouse integrin α3/CD49c antibody was from R&D System. Anti-human integrin α3 (ASC-1) blocking antibody was from Merck-Millipore. shRNA lentivirus transducing particles for human ITGA3 (clones: 002204.2-1161s21c1; 002204.1-2356s1c1 and 002204.1-2887s1c1), ITGB1 (clones: TRCN0000275133; TRCN0000275134 and TRCN0000275135), and the control pLKO.1 non-mammalian shRNA were purchased from MISSION® shRNA (Sigma-Aldrich).

### LPS-induced Lung Injury and Pulmonary Permeability Assays:

Mice were randomly divided into the following groups (n=3-5 each): vehicle only (PBS), negative control (insertless phage particles), targeted phage (CAKSMGIDVC-displaying phage particles) and positive-control (LPS-dextran treated). To induce acute lung injury, mice were anesthetized with 1% isoflurane and aerosolized with LPS (0.5 mg/kg; Klebsiella pneumoniae, Sigma) in 50 μL of PBS. Five hours later, the animals were aerosolized with 50 μL of a solution containing high molecular weight dextran (70 kDa, Invitrogen) at 10 mg/kg body weight dissolved in sterile PBS. The remaining groups were aerosolized with 50 μL of PBS only (vehicle), 10⁹ TU of insertless phage (negative control) or 10⁹ TU of targeted phage (displaying CAKSMGIDVC). The mice were sacrificed after 1 h. Thirty minutes prior to endpoint, Evans Blue dye (20 mg/kg) was administered intravenously (IV). Lungs were perfused and homogenized in PBS for Evans Blue dye extraction measurement. Tissue homogenate was quantitated at 620 nm absorbance and corrected for the presence of heme pigments. The concentration of Evans Blue dye was determined according to a standard calibration curve and expressed as total protein (μg of protein/mL). Neutrophils in BALF were counted with Trypan Blue (ThermoFisher Scientific) in a cell counter. Total protein in BALF was determined by the bicinchoninic acid (BCA) calorimetric assay (ThermoFisher Scientific). Fluorescent dextran was purchased from Invitrogen and Evans blue dye from Sigma-Aldrich. Anti-fd bacteriophage antibody was purchased from Sigma-Aldrich.

### Phage Binding Assays:

Phage binding to recombinant proteins (α3β1, α6β1, α6β4, NRP-1 and SDC-1) and BSA (Sigma) were performed as described (Cardó-Vila, et al., 2008, PLoS One 3:e3452). Briefly, 100 ng of each of the indicated proteins dissolved in 50 μL PBS were immobilized in microtiter wells overnight (ON) at 4° C. Wells were washed twice with PBS, blocked with PBS containing 3% BSA for 1 h at room temperature (RT), and incubated with targeted CAKSMGDIVC-displaying phage particles or control insertless phage particles in 50 μL of PBS containing 1.5% BSA. After 2 h at RT, wells were gently washed 10 times with PBS, and phage was recovered by host bacterial infection and represented as “Relative TU” as an empirical measure of the biological replicates and controls when compared to one another as described (Arap, W. et al., Nat. Med., 2002, 8:121-127; Cardó-Vila, et al., 2008, PLoS One 3:e3452). Human recombinant proteins α3β1, α6β1, α6β4, NRP-1 and SDC-1 were all commercially obtained from R&D Systems. Recombinant GST and CAKSMGDIVC-GST were produced in E. coli transformed with the pGEX4T-1 plasmid (Amersham, GE Healthcare) and purified with standard protocols. All synthetic peptides were custom manufactured by Merrifield synthesis and quality-controlled to the necessary specifications (Biomatik and PolyPeptide Laboratories).

### Cellular Barrier Permeability Assays:

Cells were grown on TRANSWELL® inserts (0.4 μm pore size) to complete confluence. Following equilibration (30 min, 37° C.) with pre-warmed serum-free DMEM, a dose solution containing either targeted CAKSMGDIVC-displaying phage particles or control insertless phage particles (10⁹ TU each) were each added to the upper chamber (donor) of the TRANSWELL® system. Phage particles transported across the cell monolayer were collected by the sampling of the bottom chamber (receiver) at pre-determined intervals throughout the experiment with the replacement of receiver chamber fluid with warm media. Phage particle transport was determined by TU count. shRNA lentivirus transducing particles for human ITGA3, ITGB1, and the control pLKO.1 non-mammalian shRNA were purchased from MISSION® shRNA (Sigma- Aldrich) and cell transduction was performed as indicated by the manufacturer.

### Fluorescence Microscopy Imaging:

Cells were seeded onto circular coverslips in 24-well plates (1.5×10⁵ cells/coverslip) in complete medium and grown ON at 37° C. in 5% CO₂. Cells were washed three times with PBS and fixed in PBS containing 4% paraformaldehyde (PFA) (Electron Microscopy Science) for 10 min at RT followed by incubation in 50 mM ammonium chloride buffer for 30 min and blocking solution of PBS containing 1% BSA for 1 h. For intracellular staining of phage particles, cells were blocked with DMEM containing 30% FBS at 37° C. for 1 h following by incubation with 10⁹ TU phage particles in DMEM containing 10% FBS at 37° C. for 1 h. The cells were washed five times with PBS containing 10% BSA followed by five washes with glycine buffer containing 50 mM glycine and 150 mM NaCl at pH 2.8 for three min each to remove adherent phage particles. The cells were then washed with PBS, fixed in PBS containing 4% PFA for 10 min, permeabilized with 0.2% Triton X-100 for 10 min, washed with PBS, and then blocked with PBS containing 5% normal serum and PBS containing 1% BSA for 30 min. The primary anti-fd bacteriophage (Sigma-Aldrich) and anti-mouse α3 integrin/CD49c (R&D System) antibodies were diluted in PBS containing 1% BSA, incubated with cells for 2 h at RT, washed five times with PBS, and then incubated with secondary antibodies for 1 h at RT. For nuclear staining, VECTASHIELD mounting medium containing 4′,6-diamino-2-phenylindole (DAPI, Vector Laboratories) was used. Fluorescent images were acquired on a Nikon Eclypse Ti2 inverted fluorescence microscope (Nikon). For tissue immunofluorescence, paraffin-embedded lung tissue sections (10 μm thick) were incubated with Histochoice (Sigma-Aldrich) followed by paraffin removal with xylene and ethanol. After antigen retrieval with a Dako Target Retrieval Solution (Agilent Dako) at pH 6.0, the slides were washed, blocked with 10% donkey serum in TRIS-buffered saline containing 0.1% Tween 20 (TBST) for 1 h and incubated with the antibodies: anti-proSPC (1:50) (Millipore, AB3786), anti-podoplanin (1:100) (Thermo-Invitrogen, eBio8.1.1), anti-CCSP antibody (Millipore-Merck, 07-623), anti-fd bacteriophage antibody (1:500) (Sigma-Aldrich), and anti-α3 integrin/CD49c (R&D System) followed by incubation with conjugated secondary antibodies. High-resolution images were obtained by two-photon confocal microscopy at the Advanced Light Microscopy Core Facility, University of Colorado (Denver). Pixel co-localization was analyzed with Fiji ImageJ Software.

### Phage Overlay and Tissue Immunohistochemistry:

Lung tissue sections (5 μm) were deparaffinized, rehydrated, and blocked for endogenous peroxidases and for nonspecific protein binding (Agilent Dako). For phage overlay assays, tissue sections were incubated with targeted CAKSMGDIVC-displaying phage particles or negative control insertless phage particles (2×10⁹ TU each) for 2 h at RT. After washes with TRIS-buffered saline containing 0.1% Tween 20 (TBST), the slides were incubated with the primary anti-fd bacteriophage antibody (1:800) followed by incubation with rabbit Horseradish peroxidase (HRP)-conjugated secondary antibody. Integrin α3 was detected with anti-α3 chain antibodies (1:500) followed by appropriate HRP-conjugated secondary antibodies. Images were acquired in a Nikon Eclypse Ti2 inverted microscope.

### Isolation of Lung Cells and Cell Sorting:

Six-to-eight week old female BALB/c mice were used. After aerosolization, the animals were sacrificed and perfused with 10 mL PBS followed by gentle inflation of the lungs with air. The lungs were washed with a PBS solution containing 5 mM EDTA and 5 mM EGTA, followed by RPMI medium and RPMI containing 25 mM HEPES and elastase (4.5 U/mL), and immediately followed by inflation of the lungs with a solution of water containing 1% low melting point agarose (Promega) and tissue digestion for 45 min at 37° C. After gently mincing the tissue, cells were filtered with 100 μm (Falcon #352360), 40 μm (Falcon #352340), and 20 μm (Pluriselect #43-50020-01) filters and centrifuged with a cushion layer of 150 μL of 100% Percoll (Sigma-Aldrich) in a 15 mL conical tube. For cell sorting, cells were blocked with Fc block anti-mouse CD16/CD32 antibody (BD Pharmigen, 553142) and stained with rat anti-mouse EPCAM brilliant violet 421 conjugated (BioLegend, 118225), rat anti-mouse CD45 Alexa fluor 700 conjugated (eBioscience, 56-0451-80), Syrian hamster anti-mouse podoplanin monoclonal antibody PE-Cyanine 7 conjugated (eBioscience, 25-5382-80), rat anti-CD31 FITC conjugated (BD Pharmigen, 553372) and rat anti-mouse F4/80 PE-conjugated (BD Pharmigen, 565410). Cells were sorted in an iCyt sy3200 (Sony) cell sorter previously calibrated with compensation beads (UltraComp eBeads, Thermo Scientific). AT1-, AT2-enriched cell populations and macrophages were centrifuged, and the number of phage particles per cell type was determined by TU count.

### Mathematical Modeling:

To investigate the transport of phage particles across the cellular monolayer, data from the in vitro Transwell assays (FIG. 3E) was used to quantify the parameters of an empirical mathematical model of phage accumulation in the bottom chamber (Equation 1). The mass of phage particles [N(t), measured experimentally as TU] transported across the cellular barrier at time t, was represented by Equation (Eq. 1), as follows:

N(t)=Nₛ(1−e^{-kt}) (Eq. 1)

where, Nₛ represent the mass of phage particles (TU) in the bottom chamber at saturation; k is the transport rate constant, the inverse of which gives the characteristic time (τ=1/k) of the transport process. The first derivative of Equation 1

$$( i . e . , \frac{d N ( t )}{d t} = k {N}_{s} {e}^{- k t} )$$

provides the time-dependent transport rate of phage particles across the in vitro cellular barrier. Least squares fitting of the model to the experimental data was performed in MATLAB. The transport rate decays exponentially over time at rate k, and its maximal value can be determined at time t=0 as

$$\frac{d N ( t )}{d t} {\vert}_{t = 0} = k {N}_{s} = \frac{{N}_{s}}{τ} .$$

To understand the systemic disposition kinetics of phage particles in vivo, a two-compartment pharmacokinetic model was developed (FIG. 5C). This model is based on the principles of conservation of mass and law of mass action, represented by the following system of ordinary differential equations (Eq. 2-5):

Lung airspace sub-compartment

$$\begin{matrix}\frac{d {N}_{L , a}}{d t} = - ( {k}_{m a c} + {k}_{a} ) {N}_{L , a} , & {N}_{L , a} ( 0 ) = {N}_{0}\end{matrix} \qquad ( Eq . 2 )$$

Mononuclear phagocyte sub-compartment

$$\begin{matrix}\frac{d {N}_{L , mac}}{d t} = {k}_{m a c} {N}_{L , a} , & {N}_{L , mac} ( 0 ) = 0\end{matrix} \qquad ( Eq . 3 )$$

Central compartment (blood stream)

$$\begin{matrix}\frac{d {N}_{C}}{d t} = {k}_{a} {N}_{L , a} + {k}_{2 , 1} {N}_{P} - ( {k}_{e x} + {k}_{1 , 2} ) {N}_{C} , & {N}_{C} ( 0 ) = 0\end{matrix} \qquad ( Eq . 4 )$$

Peripheral compartment (slowly perfused organs)

$$\begin{matrix}\frac{d {N}_{P}}{d t} = {k}_{1 , 2} {N}_{C} - {k}_{2 , 1} {N}_{P} , & {N}_{P} ( 0 ) = 0\end{matrix} \qquad ( Eq . 5 )$$

where, N_{L,a}, N_{C}, and N_{P} represent the mass of phage particles (TU) in the lung air space, central, and peripheral compartments, respectively; N₀ is the mass of inhaled phage; N_{L,mac} is the mass of phage particles in the alveolar macrophage sub-compartment; k₁₂ represents the first order phage transfer rate constant from the central to peripheral compartment, and k_{2,1} represents the first order phage transfer rate constant from the peripheral to central compartment. The system of ordinary differential equation (ODE) was solved numerically as an initial value problem in

MATLAB using the built-in non-stiff ODE solver ode45, and then least squares fittings of the model to the in vivo data were performed in MATLAB

### Intratracheal Administration of CAKSMGDIVC-displaying Phage Particles:

Phage input in mice was 10⁹ TU of the targeted CAKSMGDIVC-displaying phage particles or negative control insertless phage per mouse (n=4 each group) administered via the intratracheal route with 50 μL of PBS with a MicroSprayer® Aerosolizer coupled to a high-pressure syringe (Penn-Century) and a small animal laryngoscope (Penn-Century). Two serial doses of intratracheal aerosol administration with targeted CAKSMGDIVC-displaying phage particles or control insertless phage particles were performed in paired rhesus monkeys. Because of species-specific differences in the respiratory tract anatomy from mice to non-human primates and to humans, as well as body size, breathing patterns, inhalation methods, and device, the dose of phage administration was increased to 10¹² TU per individual monkey. The monkeys were anesthetized using an intramuscular injection of Telazol (tiletamine and zolazepam) for induction, followed by endotracheal intubation and maintenance on inhaled isoflurane (the percentage was adjusted based on monitoring of anesthetic depth-heart rate, respiratory rate, response to stimuli). Sterile IV tubing was cut to a length approximately 4 cm longer than the endotracheal tube and was attached to a syringe containing CAKSMGDIVC targeted phage or the control insertless phage. The tubing was inserted down into the endotracheal tube and phage was slowly administered into the trachea over 60 seconds. For the blood collections, monkeys were anesthetized with an intramuscular injection of ketamine. At dose 1 administration (set as day 1), blood samples were collected hourly up to 6 h post-aerosolization. The second dose was administered after 28 days while serum collection was performed every 14 days. Endotoxin removal was performed for each phage preparation and before the administration of each intratracheal dose. Phage solutions containing endotoxin were treated with 10% Triton X-114 in endotoxin-free water on ice for 10 min. The solution was then warmed to 37° C. degrees for 10 min followed by removal of the Triton X-114 phase by centrifugation at 14,000 rpm for 1 min. The levels of endotoxin were measured using the Limulus Amebocyte Lysate (LAL) Kinetic-QCL kit from Lonza. Phage preparations with <0.05 EU/mL of endotoxin were used in this study.

### Serological Analysis in Non-human Primates:

IgG from monkeys were purified from the serum with Protein G agarose resin (Sigma Aldrich). The flow-through was used to purify IgA with Jacalin agarose resin (Thermo Fisher Scientific). ELISA was performed with 10¹⁰ particles/50 μL coated onto 96-well plates ON at 4° C. (Nunc MaxiSorp flat bottom, Thermo Scientific). For this assay, phage titration was performed by quantitative qPCR with fUSE primers (fUSE5 forward as follows: 5′-TGAGGTGGTATCGGCAATGA-3′ and fUSE5 reverse: 5′-GGATGCTGTATTTAGGCCGTTT-3′). ELISA was also performed with 96-well plates coated with the synthetic peptide (10m/mL) CAKSMGDIVC or an unrelated control peptide (CGRRAGGSC) unless otherwise specified overnight at 4° C. Coated plates were blocked with PBS containing 5% low-fat milk and 1% BSA (Sigma-Aldrich) for 1 h at 37° C. Two-fold serial dilutions (starting at 1:4) of purified IgG and IgA were applied to the wells and incubated for 2 h at 37° C. Following three washes with PBS and PBST, bound antibodies were detected with an anti-monkey IgG (KPL; 074-11-021) or IgA (KPL: 074-11-011) HRP-conjugated. Purified polyclonal IgG anti-CAKSMGDIVC antibodies (Biomatik USA, Delaware) and anti-fd bacteriophage antibody (Sigma) served as positive controls. Plates were read at 450 nm absorbance.

### Statistical Analysis:

Differences between groups were tested for statistical significance with Student's t-test or analysis of variance (two-way ANOVA). Statistical significance was set as p<0.05. The analyses were performed in GraphPad Prism 8 and MATLAB R2015b.

### Selected Results

Inhalation-based vaccination to achieve rapid immunization, particularly in developing countries and disaster areas, is needle-free and, unlike the oral route, not subject to undesirable first-pass metabolism. The lung surface area varies by measurement techniques and degree of inflation and estimates may fluctuate from 70 to 130 square meters in an inflated lung. The thin and highly permeable alveolar region of the lung, comprised of alveolar epithelial type-1 (ATI) and type-2 (AT2) cells, and the associated microvascular endothelium, generally defines the selective permeability of molecules allowed to cross into the blood stream. Low molecular weight drugs, peptides, or proteins such as insulin, small viruses, and even immunogens are among suitable candidates for inhaled agent administration. More recently, inhalation-based vaccination platforms have gained particular attention for effective field use and protection against airborne pathogens such as tuberculosis, influenza, Ebola virus, and measles; indeed, the ongoing pandemic of coronavirus disease (COVID-19) caused by severe acute respiratory syndrome coronavirus 2 (SARS-CoV-2) provides prima fade evidence for the magnitude of an unmet public health need in the setting of a global pandemic.

In theory, pulmonary delivery improves therapeutic bioavailability while reducing potential side effects by achieving a more rapid onset of action; however, inhalation also poses inherent challenges, particularly for systemic applications, restricting its use at this point in time to respiratory diseases. In general, inhalation-based therapies are assessed through the monitoring of the pharmacological endpoints in vivo. Yet surprisingly, the actual mechanisms of how inhaled particles interact with the air-blood barrier, the physicochemical changes in the molecules in contact with the pulmonary surface, bioavailability, uptake by local immune system and clearance processes or removal of insoluble active compounds remain largely unknown.

In certain embodiments, lipophilic moieties can be rapidly absorbed through the lungs by passive diffusion across the alveolar cell plasma membrane, while hydrophilic moieties tend to be transported by specific surface receptors or through cellular tight junctions. Uncovering physiological mechanisms that allow selective transport of active particles through the lung while preserving respiratory function and homeostasis is important to the design of a general pulmonary delivery system for multiple applications.

As described in the present disclosure, a phage display-based combinatorial random peptide platform was devised and applied to uncover unique ligand/receptor-mediated pulmonary transport pathways for safe and effective absorption of particles across the air-blood barrier towards targeted phage-based applications, including but not limited to vaccines.

In one aspect, an aerosol phage display random peptide library was screened in vivo to select and isolate targeting peptides capable of crossing intact lung air-blood barriers into the blood stream. A new ligand peptide motif, CAKSMGDIVC, was validated and its corresponding receptor, the integrin α3β1, was biochemically purified via affinity chromatography. Integrin α3β1 is expressed on the surface of alveolar epithelial cells as well as club cells, the epithelial secretory cells found in the terminal and respiratory bronchioles of the lung. Specific binding of targeted phage particles displaying the CAKSMGDIVC motif to α3β1 was found to promote phage particle uptake and transport to the circulation in vivo. In another aspect, a two-compartment pharmacokinetic mathematical model was developed to understand and predict the systemic disposition kinetics of phage particles upon targeted pulmonary administration. In yet another aspect, the ligand/receptor-based aerosol system was evaluated in a non-human primate model where the system was found to be useful for pulmonary-targeted delivery and potential development of phage-based vaccines. Together, the combinatorial selection system and findings reported herein provide a versatile enabling platform for ligand-directed pulmonary aerosol delivery with broad translational applications.

### Example 1: Serial Screening of Phage Particles that Cross the Intact Pulmonary Barrier In Vivo

Identification of ligand peptide motifs that would be physiologically transported across the intact lung epithelium was implemented by administering a phage display library (10⁹ unique sequences) comprised of ˜10¹⁰ transducing units (TU) of random cyclic peptides by intratracheal aerosolization in vivo in mice. This method utilizes a micro sprayer aerosolizer of an aqueous preparation, based on high-pressure generation of particles with less than 2.5 μm in size that are expected to reach the distal airspaces, as shown in preclinical studies of pulmonary drug deposition (Guillon, et al., 2018, Int J Pharm 536:116-126). In the initial round of selection, passage through the lung barrier in mice was confirmed by the detection of phage particles in blood samples collected at fixed time points and up to six hours post-aerosolization (FIG. 1A). The in vivo screening was performed in serial cohorts of mice (n=3 each). After each subsequent round of selection, recovered phage particles were pooled, amplified, and re-aerosolized (FIG. 1B); to select ligand peptides that efficiently mediate transport of phage particles across the pulmonary epithelium-endothelium layers, time-to-collection was reduced stepwise from 60 min in Round 1 (R1) to a 5 min in Round 4 (R4) (FIG. 1B). Progressive enrichment was observed (FIG. 1C) and the corresponding DNA encoding individual peptides recovered from R4 were sequenced. The percentage of each enriched peptide is depicted; notably, only four dominant peptides comprised nearly half of the total number of sequences whereas the other half of the sequences (n=16) were below the 5% in frequency arbitrarily set as an experimental threshold for further research and development in this work (FIG. 1D). When these peptide-displaying phage particles were individually administered via the airspaces to mice, all four dominant ligand candidates crossed the pulmonary barrier and reached the systemic circulation within 1 h post-administration, as opposed to insertless phage particles (range 50-200-fold; mean ˜110-fold), which served as a negative control (FIG. 1E).

Without wishing to be limited by any theory, functional analysis was focused on the index ligand peptide sequence CAKSMGDIVC (phage clone 2) because it showed one of the highest transport efficiencies to the systemic circulation (FIG. 1E). Indeed, the CAKSMGDIVC-displaying phage particles peptide were most efficiently transported through the lung and present in the bloodstream at very high concentrations up to 2 h post-aerosolization, whereas non-targeted (insertless) negative control phage particles were barely detectable (FIG. 1F). Phage clearance from the blood stream was observed 8 h post-aerosol administration. Without wishing to be limited to any theory, this may take place through a non-specific clearance mediated by the reticuloendothelial system (Staquicini, F. I. et al., J Clin Invest., 2011, 121:161-173; Pasqualini, R. et al., Nature, 1996, 380:364-366; Hajitou, A. et al., Cell, 2006, 125:385-398). Together, these data indicate that CAKSMGDIVC-displaying phage particles are deposited and selectively transported from the lung into the systemic circulation. Thus, phage uptake and transport of the present peptide-displaying phage particles is likely mediated by specific ligand-receptor interactions.

### Example 2: Distribution, Clearance and Lung Homeostasis upon Peptide-mediated Phage Transport

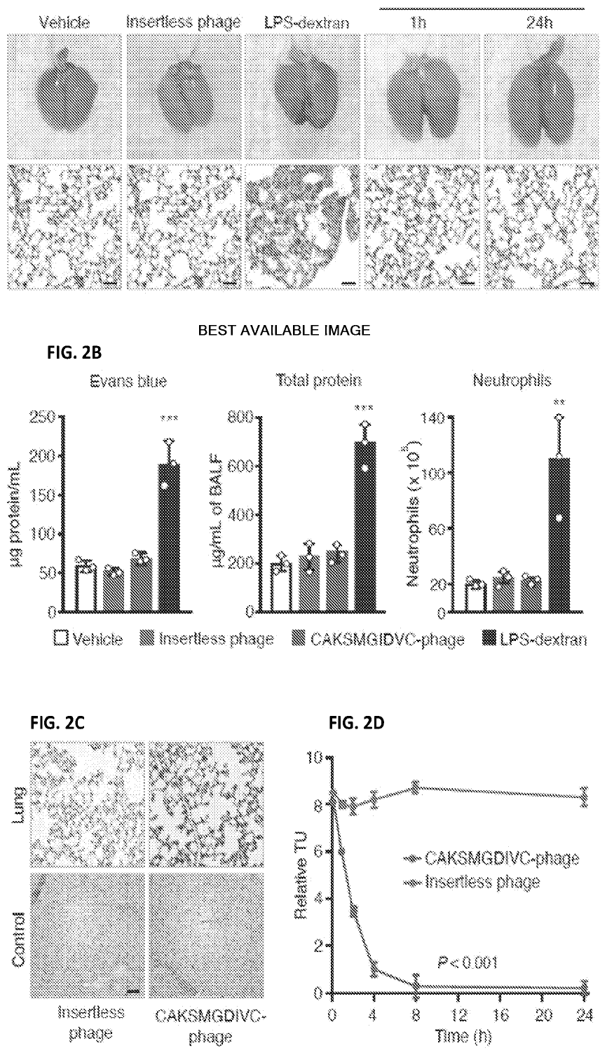

To rule out the possibility that the enhanced transport of the index targeted phage particles was caused by tissue damage induced during intratracheal aerosolization, the morphology and homeostasis of lungs from animals aerosolized with CAKSMGDIVC-displaying phage particles in comparison to those aerosolized with vehicle only or the negative control insertless phage particles (FIGS. 2A-2D) were evaluated. No detectable evidence of pulmonary tissue damage (including lung edema and/or inflammation) was observed by gross morphology or histopathology up to 24 h post-aerosolization (FIG. 2A). Lung permeability was evaluated by quantification of Evans Blue extravasation (an azo dye that has a very high-affinity for serum albumin) in the lung tissue or protein content in the bronchoalveolar lavage fluid (BALF) upon lung injury. No detectable differences were observed between targeted and control insertless phage particles. Furthermore, no evidence of acute inflammation was detected by neutrophils count in the BALF of mice treated with CAKSMGDIVC-displaying phage particles or negative control insertless phage particles.

The data was then compared with aerosol phage particles with those of a paradigmatic inducer of lung injury, lipopolysaccharide (LPS) followed by a high-molecular weight dextran (hereafter referred to as LPS-dextran) administered via aerosol, as a positive control for tissue damage characterized by leukocyte infiltration and extravasation of vascular fluids. In LPS-dextran treated mice, alveolar damage, marked vascular congestion, and microvascular injury were confirmed by gross morphology and histopathology (FIG. 2A). In addition, mice treated with LPS-dextran (positive control) showed clear signs of marked lung injury, impaired integrity of pulmonary tissue, and marked neutrophil infiltration (FIG. 2B).

In certain non-limiting embodiments, transport of the index targeted phage from the lung into the bloodstream can be mediated by specific binding of the ligand CAKSMGDIVC to cell surface receptor(s). A series of phage binding assays were designed to evaluate binding of the CAKSMGDIVC-displaying phage particles to target cells in vitro and in vivo. First, a phage overlay assay (Staquicini, et al., 2011, Proc Natl Acad Sci USA 108:18637-18642) was used to show phage binding to cells on lung tissue sections (FIG. 2C), whereas no binding was detected on tissue sections from a control organ (shown is pancreas). Control insertless phage particles showed only background staining (FIG. 2C).

Finally, binding and transport of targeted and control phage particles into the bloodstream as a function of time was quantitated in the lung of aerosolized mice (FIG. 2D). A marked reduction in the number of CAKSMGDIVC-displaying phage particles present in the lung was observed starting at 1 h and continuing up to 8 h post-aerosolization. After 24 h, very few targeted phage particles were still detected in the lung (FIG. 1F). In contrast, levels of negative control insertless phage particles remained unaltered during the same time frame. These results recapitulate the kinetics of CAKSMGDIVC-displaying phage particles transport into the systemic circulation (FIG. 1F), which showed high amounts of phage particles in the bloodstream between 1 h and 4 h post-aerosolization. Phage clearance from the bloodstream was observed 8 h post-aerosolization. Without wishing to be limited by any theory, this can happen through the reticuloendothelial system non-specific clearance. Together, these data indicate that CAKSMGDIVC-displaying phage particles are deposited and selectively transported from the lung into the systemic circulation.

### Example 3: Receptor Identification and Validation In Vitro

Peptide affinity chromatography was used to identify the corresponding candidate receptor(s) targeted by the CAKSMGDIVC ligand. Total protein extracts from a human lung adenocarcinoma cell line (A549 cells) were loaded onto a CAKSMGDIVC peptide-conjugated column and interacting proteins were eluted with an excess amount of soluble CAKSMGDIVC synthetic peptide (produced through Merrifield synthesis). Eluted proteins were subsequently identified by mass spectrometry (Table 1).

Five main potential receptor candidates were selected: the integrins α3β1, α6β1, and α6β4, as well as neuropilin-1 (NRP-1) and syndecan-1 (SDC-1). Cell-free binding assays to immobilized recombinant proteins in vitro showed that targeted CAKSMGDIVC-displaying phage particles bound preferentially to α3β1 integrins in comparison to the other receptor candidates (FIG. 3A). Bovine serum albumin (BSA) and insertless phage served as negative controls and showed binding only at minimal background levels. Competition assays in the presence of increasing molar concentrations of either the targeted synthetic peptide or an unrelated negative control synthetic peptide (sequence CGRRAGGSC; Cardó-Vila, M. et al., PLoS One, 2008, 3:e3452) confirmed the binding specificity of CAKSMGDIVC-displaying phage to α3β1 integrins (FIG. 3B).

Interaction with endogenous α3β1 integrins expressed on the surface of human alveolar epithelial adenocarcinoma cells (A549) was evaluated. The presence of α3β1 integrins on A549 cell surface has been reported and was also confirmed by immunofluorescence (FIG. 3C). The Biopanning and Rapid Analysis of Selective Interactive Ligands (termed BRASIL) methodology (Giordano, et al., 2001, Nat Med 7:1249-1253) was used to demonstrate binding of the CAKSMGDIVC-displaying phage particles to A549 cells. No binding above background was observed with the control insertless phage particles (FIG. 3D).

Uptake and transport of CAKSMGDIVC-displaying phage particles through cell monolayers was evaluated. A549 cells were seeded on the upper chamber of transwell chambers and exposed to either CAKSMGDIVC-displaying phage particles or the control insertless phage particles. Phage transport from the upper chamber and across the A549 cell monolayer was determined by TU count recovered from the bottom chamber. Transport of targeted CAKSMGDIVC-displaying phage particles was detected as early as 1 h following addition, with the highest accumulation occurring from 8 to 24 h (FIG. 3E). Minimal transport of insertless phage particles was observed throughout the experiment. The integrity of the cell monolayer was not affected by any of the targeted or control phage particles, as demonstrated by the absence of fluorescent dextran transport (Table 2). Finally, A549 cells were genetically depleted of α3 or β1 integrin chain to confirm binding specificity. The knockdown of α3β1 integrin was obtained by transducing A549 cells with shRNA lentiviral particles targeting the human ITGA3 gene that encodes for the α3 integrin chain and the human ITGB1 gene that encodes for β1 integrin chain. Untargeted shRNA (pLKO) lentivirus particles were used as control (FIGS. 7A-7B). Targeted phage binding, internalization, and transport were markedly reduced in α3 integrin chain-silenced A549 cells, whereas no effect was observed in cells transduced with the negative control shRNA (FIG. 3F). Only partial binding inhibition was observed when the β1 integrin chain was silenced (FIG. 3G). Biochemically, competition assays with either a recombinant CAKSMGDIVC-GST peptide (FIG. 3H) or with anti-α3 integrin chain antibodies (FIG. 3I and FIG. 7C) confirmed concentration-dependent ligand-receptor specificity and suggested that the binding of the CAKSMGDIVC-displaying phage particles might target a site within the α3 chain of α3β1 integrin heterodimer.

To characterize the receptor-mediated phage transport process, an empirical mathematical function (Equation 1) was fitted to the in vitro Transwell data and determined model parameters of phage particles transport across the cell monolayer (FIG. 7D). Strong correlation between the model fits and their corresponding experimental data were observed, as assessed by the Pearson correlation coefficient (R>0.96 for both cases), thus providing confidence in the mathematical model. The characteristic time τ of the transport process is smaller for the control insertless phage than for the targeted CAKSMGDIVC-displaying phage (˜1.5 h vs. ˜4.8 h) as shown (Table 3). Unlike the insertless phage, targeted CAKSMGDIVC-displaying phage performs an additional step of engaging with α3β1 integrins to cross the cellular monolayer, which likely explains the longer characteristic time for its transport. However, specific targeting allows a greater number of phage particles Nₛ to cross the cellular barrier. Combining the two-model parameters τ and Nₛ, the overall transport process can be characterized by the initial rate of transport, and it has a value that is about four orders of magnitude greater for targeted CAKSMGDIVC-displaying phage particles than for the control insertless phage particles (Table 3). Together, the data indicate that CAKSMGDIVC-displaying phage particles bind and are transported across cells monolayer by a receptor-dependent mechanism mediated by α3β1 integrins.

### Example 4: CAKSMGDIVC-displaying Phage Particles Target α3β1 Integrins In Vivo

Having α3β1 integrin identified as the corresponding membrane receptor specifically mediating the observed peptide-induced transport of CAKSMGDIVC-displaying phage particles in cell-free and in cell-based assays, immunohistochemistry and immunofluorescence were used to study the cellular expression and tissue localization of α3β1 in lung tissue sections. The presence of α3β1 integrins was detected in cells in the airways and alveolar regions (FIGS. 4A-4B, 4E and FIG. 8A) of the lung. In particular (FIG. 4A), the expression of α3β1 integrin (red) was detected in type-1 (AT1, purple) and type-2 (AT2, green) lung alveolar epithelial cells, and cells of the respiratory bronchioles (FIG. 4B). Although some variation in the levels α3β1 integrin was noticed detected by immunofluorescence analysis, the presence of α3β1 integrin was confirmed in these cell populations by single-cell RNA sequencing (scRNA-seq) on mouse lung tissue. Transcriptomic analysis of flow-cytometry sorted cell populations confirmed that Itga3 and Itgb1 transcripts encoding for mouse α3β1 integrin are present in basal cells, airways epithelial ciliated and non-ciliated cells as well as alveolar epithelial cells, and to a higher degree in AT1 (˜6-fold more Itga3 and ˜2-fold more Itgb1 than AT2 cells) (FIG. 8B). Since AT1 cells cover over 95% of the alveolar surface, their high expression of α3β1 integrin can facilitate efficient phage transport across the lung tissue and into the bloodstream.

Tests were performed to determine whether α3β1 integrin-expressing cells were indeed implicated in the transport of the CAKSMGDIVC-displaying phage particles across the pulmonary barrier in vivo. Either targeted or control phage particles were administered via aerosol and, after 1 h, mice were sacrificed and perfused through the heart with phosphate- buffered saline (PBS). The lungs were fixed, embedded, and sectioned for immunofluorescence analysis. Lung tissue sections from mice administered via aerosol with either CAKSMGDIVC-displaying phage particles or control insertless phage particles were immunostained with specific markers for each cell population and with an anti-phage antibody. Confocal microscopy analysis shows that CAKSMGDIVC-displaying phage particles target alveolar epithelial AT1 and AT2 in the alveoli. Co-localization of CAKSMGDIVC-displaying phage particles with AT1 cells (purple) and AT2 cells (green) are indicated by white arrows (FIG. 4C) and the relative quantification is also represented (FIG. 4D); only background staining of the control insertless phage particles to alveolar cells was observed. Notably, not all AT1 and AT2 cells were positive for phage staining. While not wishing to be limited by theory, this suggests that the variation in α3β1 integrin expression might determine the binding and transport of CAKSMGDIVC-displaying phage particles in these cell populations. High concentrations of targeted CAKSMGDIVC-displaying phage particles or control insertless phage particles were detected in macrophages (yellow arrows, FIG. 4C).

Marked expression of α3β1 integrin was also observed in cells of bronchioles (FIG. 4B, white arrows) mainly non-ciliated club cells (FIG. 4E). In mice, ciliated and non-ciliated cells of the lung are the primary constituents of the bronchioles. Club cells are the main source of the club cell secretory protein (termed CCSP) into the extracellular fluid lining the airspaces. By using a specific antibody against CCSP, it was confirmed that club cells constitute most of the cells expressing α3β1 integrin in the bronchioles. Also, high colocalization of CAKSMGDIVC-displaying phage particles, relative to negative control insertless phage particles and club cells, was present in the bronchiolar region and again confirmed with an antibody against CCSP (FIG. 4F). Taken together, these experimental results establish that CAKSMGDIVC-displaying phage particles bind to α3β1 integrin Expressed on the plasma membrane of AT1 and AT2 alveolar epithelial cells and club cells.

### Example 5: CAKSMGDIVC-displaying Phage Particles Target α3β1 Integrin In Vivo

To further evaluate the bioavailability of CAKSMGDIVC-displaying phage particles after aerosol administration, the binding capabilities of the phage particles to specific lung cells were determined by flow cytometry. Following aerosol of targeted and non-targeted phage particles, the lungs were harvested and digested into a collection of single cells. Specific cell populations of the lung were isolated and flow cytometry sorted in three main cell populations (FIG. 5A): AT1-enriched population (EPCAM⁺, CD45⁻, CD31⁻, T1α^{high}); AT2-enriched population (EPCAM⁺, CD45⁻, CD31⁻, Tα^{low}) and mononuclear phagocytes (EPCAM⁻, CD45⁺, CD31⁻, F4/80⁺). The number of either targeted or control phage particles bound to each cell population was determined by TU counting after infection with a host bacterium. CAKSMGDIVC-displaying phage particles were recovered from both AT1- and AT2-enriched populations; binding to AT1-enriched population was ˜2-fold higher than binding to AT2-enriched cells. These results are also consistent with the scRNA-seq analysis in which transcripts for α3β1 integrin are ˜2-fold higher in AT1 than AT2 cells (FIG. 8B). Negative control phage particles showed only background binding to both cell populations. High amounts of either targeted or control phage were recovered from the mononuclear phagocyte-enriched cell population, a result in agreement with non-specific phagocytosis within the lung airspaces (FIG. 5B).

A non-limiting two-compartment pharmacokinetic model was developed (FIG. 5C) to understand and predict the in vivo disposition kinetics of phage particles that involves transport from the lungs into the systemic circulation, and clearance by the mononuclear phagocyte system (MPS). The non-limiting model consists of (i) the systemic blood pool and rapidly perfused organs (designated as the central compartment) and (ii) slowly perfused organs, i.e. fat and muscle (designated as the peripheral compartment). The transport of phage particles from the lung airspace into the systemic blood pool (i.e., the central compartment) is characterized by the first order absorption rate constant kₐ. The lung airspace includes the mononuclear phagocytes population capable of internalizing phage particles at a rate characterized by the first order macrophage uptake rate constant k_{mac}. The central and peripheral compartments exchange phage particles at rates characterized by the first order transfer rate constants k₁₂ and k₂,₁. Finally, the clearance of particles from blood by the hepato-splenic route or MPS is characterized by the first order excretion rate constant kₑₓ. The pharmacokinetic model is based on the principles of conservation of mass and law of mass action, represented by the system of ordinary differential equations (Equations 2-5). As shown in the FIG. 5D, the model was fit to the data corresponding to phage distribution (lung and blood) and the estimated kinetic parameters are shown (Table 4). The strong correlation (R>0.99, P<0.0001) between mathematical model fits and experimental observations confirms the modeling approach and provides confidence in the kinetic parameter estimates (FIG. 5E). The systemic bioavailability of the CAKSMGDIVC-displaying phage particles is about two orders of magnitude greater than the control insertless phage, as quantified by the area under the curve (AUC_{0-inf}) of the central compartment kinetics curve. Further, as predicted by the model, both targeted and non-targeted phage particles are rapidly cleared from the systemic circulation due to sequestration in the MPS organs (e.g., liver, spleen) as shown (Table 4; FIG. 8C). Given that the major fraction of the control insertless phage remains confined to the lung compartment, the presence of CAKSMGDIVC-displaying phage particles in the blood stream (i.e., central compartment) and slowly perfused organs (i.e., peripherical compartment) is at least one order of magnitude greater as shown (FIG. 5E; FIG. 8C), confirming the superior systemic bioavailability of targeted CAKSMGDIVC-displaying phage particles upon pulmonary administration.

### Example 6: CAKSMGDIVC Promotes Transport of Targeted Phage Particles in Non-human Primates and Elicits a Systemic, Robust, and Specific Humoral Response

Given that ligand-mediated transport of the CAKSMGDIVC-displaying phage particles across lung barriers was efficient and safe in mice, the translational experiments were expanded to a large animal model in order to validate an aerosol phage-based application for immunization towards a vaccination strategy. A non-limiting goal of this approach was to explore the unique mechanisms underlying the attributes of the CAKSMGDIVC ligand peptide, and its functional interaction with the corresponding receptor α3β1 integrin expressed on lung epithelial cells, to develop a targeted immunization system based on aerosol delivery. In certain embodiments, ligand-directed phage particles can target lymph nodes preferentially to induce specific systemic humoral responses.

To design a vaccination protocol in non-human primates, the earlier analysis was expanded to determine the humoral response in the lung and the systemic circulation in mice. After 14 days of phage aerosol administration, an overall increase in IgG, IgA, and IgM immune response reactive against phage particles was observed in the serum and the BALF of mice which were administered CAKSMGDIVC-displaying phage particles. This increase was seen relative to the controls, pre-immune or mice immunized with insertless phage particles (FIG. 10). Given that ligand-mediated transport of the CAKSMGDIVC-displaying phage particles across lung barriers was safe in mice, the vaccination protocol was applied to rhesus macaques (Macaca mulatta), a well-known species of Old World monkeys, as an experimental model far more reminiscent of human patients in going forward.

A pre-clinical trial protocol for immunization of rhesus monkeys to mimic phage aerosolization was designed, and it included administration of two serial doses of 10¹² TU of either targeted CAKSMGDIVC-displaying phage particles or control insertless phage particles through the intratracheal route. The schedule of the immunization is depicted in FIG. 6A. The presence of α3β1 integrins in lung tissue sections from rhesus monkeys was confirmed by confocal microscopy (FIG. 6B). AT1 cells were identified by positive protein staining of the Receptor for Advanced Glycation Endproducts (RAGE) which is abundantly expressed on alveolar epithelial cells. α3 integrin chain and RAGE were co-localized throughout the AT1 cells (FIG. 9A). Additionally, expression of α3β1 integrins in alveolar and airway epithelial cells on lung tissue sections from healthy human patients was confirmed by immunohistochemistry (FIG. 9B), a result supportive of the translational efforts for this technology.

Each dose was administered every 28 days and serum was collected every 14 days. The transport of CAKSMGDIVC-displaying phage particles across lung barriers into the systemic circulation was demonstrated in blood samples collected hourly after the first dose. CAKSMGDIVC-displaying phage particles were first detected in the peripheral blood 3 h post-administration, with further accumulation up to 5 h. A decrease in phage particles was observed at the 6 h time point, as determined by TU (FIG. 6C). In clear contrast, negative control insertless phage particles were detected only at minimal levels in the bloodstream at all time points. Without wishing to be limited by any theory, the transport of targeted CAKSMGDIVC-displaying phage particles across the pulmonary barrier towards the bloodstream can improve the systemic immune response. Analysis of antibody response by ELISA indicated that targeted CAKSMGDIVC-displaying phage particles generated higher titers of phage specific IgG serum antibodies than control insertless phage starting at day 28 post-administration and markedly increasing after the second dose, at days 42 and 56 post-administration (FIG. 6D).

To evaluate the extent of the antibody response generated by the pulmonary transport of CAKSMGDIVC-displaying phage particles, IgG and IgA serum antibodies were further analyzed and represented as fold change in titer. The humoral response generated by the intratracheal administration of targeted CAKSMGDIVC-displaying phage particles or the control insertless phage relative to the baseline was compared. At day 28, phage specific IgG antibody response induced by the targeted CAKSMGDIVC-displaying phage particles showed a ˜6,000-fold increase in titer relative to the baseline (FIG. 6E). This represents ˜4-fold increase in phage specific IgG titer relative to the control insertless phage (˜1,000-fold over the baseline) (FIG. 6E and FIG. 9C). Administration of a second dose of CAKSMGDIVC-displaying phage particles resulted in a substantial increase in antibody levels by days 42 and 56, with the highest difference observed at day 56 post-administration when the mean titer of phage specific serum IgG antibodies was ˜200,000-fold higher than the baseline with a sustained ˜4-fold difference over the insertless control phage (FIG. 6E and FIG. 9C).

Similar results were observed for IgA serum antibodies. Administration of CAKSMGDIVC-displaying phage particles generated higher titers of phage specific IgA serum antibodies starting at day 14 post-administration and increased considerably after the second dose, by days 42 and 56 (FIG. 6F). At day 28, the mean titer of IgA antibodies shows an increase of ˜50-fold relative to the baseline (FIG. 6G) and ˜3-fold higher than the insertless control phage (FIG. 9D).

The observation that targeted CAKSMGDIVC-displaying phage particles generated a strong and specific systemic humoral response was further demonstrated with the detection of CAKSMGDIVC-specific IgG and IgA serum antibodies by ELISA using its cognate synthetic peptide and a control unrelated peptide. Detection of CAKSMGDIVC-specific IgG and IgA serum antibodies was observed starting at day 14 for serum IgG (FIG. 6H), and at day 28 for serum IgA (FIG. 6I). Either CAKSMGDIVC-specific IgG and IgA antibodies showed ˜8-12-fold increase in titer relative to the baseline (FIGS. 9E-9F), with no substantial changes in titer after the second dose. Only minimal background cross-reactivity was detected against an unrelated synthetic negative control peptide. In certain non-limiting embodiments, there is low mass representation of the recombinant minor coat protein that encodes for the peptide sequence (CAKSMGDIVC), given that there are only about 3-5 copies of pIII per phage particle and tend to be surpassed by proteins with high copy numbers (e.g. the major coat protein pVIII, estimated at several hundred copies). In certain embodiments, specificity of the antibody response against the peptide indicates epitope recognition and activation of specific cellular immune response. Together, these results indicate that the selective transport of CAKSMGDIVC-displaying phage particles across the air-blood barrier towards the bloodstream markedly increases the specific humoral response against the phage particles and its selected ligand peptide and it thus represents an advance in the development of aerosol phage-based vaccines.

In certain embodiments, these findings support the utility and efficiency of the ligand CAKSMGDIVC peptide in targeted pulmonary delivery for multiple applications and reveal a new molecular mechanism of lung epithelium-endothelium transport mediated by internalizing α3β1 integrins.

TABLE 1
Candidate receptors identified by mass spectrometry.

| Candidates | Score | Matches | Sequences |
|---|---|---|---|
| Integrin α3 | 26932 | 1022 | 42 |
| Integrin β1 | 17234 | 976 | 34 |
| Integrin α6 | 7430 | 733 | 39 |
| Integrin β4 | 6900 | 511 | 38 |
| Neuropilin 1 | 5372 | 327 | 58 |
| Syndecan-1 | 5320 | 267 | 71 |
| Filamin B | 2100 | 432 | 63 |
| Cadherin-1 | 1749 | 197 | 28 |
| Lamin B1 | 1544 | 180 | 13 |

TABLE 2
Cell integrity upon transport of targeted or control phage particles. A549 cell barrier integrity

| Time (h) | CAKSMGDIVC-phage | Insertless phage |
|---|---|---|
| 0 | 15 ± 0.1 | 16 ± 0.5 |
| 1 | 18 ± 0.3 | 14 ± 0.6 |
| 2 | 22 ± 0.9 | 21 ± 0.6 |
| 4 | 25 ± 0.8 | 19 ± 0.5 |
| 8 | 16 ± 0.4 | 18 ± 0.8 |
| 24 | 16 ± 0.8 | 16 ± 0.5 |

| | |
|---|---|
| No cell barrier (0.1% Triton) | 5900 ± 8 |
| No cell barrier (no cells) | 6100 ± 6 |
| Media | 19 ± 0.1 |

TABLE 3
Phage particles transport across cell monolayer obtained from an empirical mathematical modeling

| | Transport rate | Characteristic of transport | Particle measure | Initial rate of |
|---|---|---|---|---|
| | constant | time τ | at saturation Nₛ | transport |
| Group | k (h⁻¹) | (h) | (TU) | (TU · h¹) |
| Insertless phage | 0.67 | 1.5 | 1.45 × 10³ | 9.72 × 10² |
| CAKSMGDIVC- | 0.21 | 4.8 | 4.6 × 10⁷ | 9.66 × 10⁶ |
| phage | | | | |

TABLE 4
In vivo pharmacokinetics of phage particles obtained from the mathematical modeling analysis

| | | | | | | AUC_{0-Inf} |
|---|---|---|---|---|---|---|
| Group | kₐ (h⁻¹) | k_{mac} (h⁻¹) | kₑₓ (h⁻¹) | k_{1,2} (h⁻¹) | k_{2,1} (h⁻¹) | (TU · h) |
| Insertless phage | 3.75 | 16.67 | 4.04 × 10³ | 889.3 | 0.13 | 7.86 × 10³ |
| CAKSMGDIVC- | 0.51 | 0.018 | 2.22 × 10³ | | | 3.59 × 10⁵ |
| phage | | | | | | |

### Selected Comments

Aerosol-based administration routes have been developed over the past several years but have achieved only relatively modest adoption perhaps because of the lack of mechanistic insight regarding fate and biodistribution, and potentially unfavorable pulmonary side effects of untargeted aerosol agents. As described in the present disclosure, an unbiased combinatorial approach was used to identify and to validate a ligand-directed pulmonary delivery system that successfully induced systemic effects with no detectable lung damage. A unique and specific role of α3β1 integrins was shown as well as the application of predictive mathematical modeling for the uptake and transport of targeted phage particles displaying a new index ligand peptide (i.e., CAKSMGDIVC) across pulmonary barriers and into the blood stream in vivo. As a proof-of-concept, an aerosol phage-based application was tested in non-human primates as an initial step towards the development of aerosol phage-based vaccines for human patient applications. This targeted method of pulmonary delivery of a highly stable and immunogenic antigen carrier (i.e., a viral phage particle) elicited a robust and specific humoral immune response, with field applications for, in non-limiting manner, vaccine and/or other therapeutic developments.

To obtain mechanistic insights and explore the diversity of surface receptors implicated in physiological transport of molecules across the air-blood barrier, a combinatorial screening of an aerosol phage display random peptide library was performed in mice. From the pool of peptide-displaying phage particles recovered from the blood stream, four dominant ligand peptide candidates mediated phage transport across the pulmonary barrier. Of these selected ligands, the index peptide CAKSMGDIVC showed one of the highest transport efficiencies in vivo, data suggesting that a specific ligand-receptor interaction likely accounts for targeted pulmonary delivery. The distribution, transport, and clearance of CAKSMGDIVC-displaying phage particles deposited in the airways upon aerosol administration were monitored in vivo and ex vivo and indicated that phage transport posed no detectable lung injury without either anatomic or physiological pulmonary impairment, results supporting that phage particles are suitable for safe inhaled human administration.

To identify the receptor(s) for the ligand CAKSMGDIVC peptide, a series of phage binding assays were performed in vitro and in vivo. Specific binding to a human recombinant α3β1 integrin followed by the functional binding and transport of CAKSMGDIVC-displaying phage particles across cell monolayer of an alveolar epithelial surrogate confirmed the ligand-receptor interaction. Evidence that targeted phage particles' crossing of the pulmonary barrier is mediated through a ligand-receptor mediated mechanism was unequivocally established by the specific binding of CAKSMGDIVC-displaying phage particles to α3β1 integrins on the surface of pulmonary AT1, AT2, and club cells in vivo. Although the expression of α3β1 in club cells is high, their functional involvement in phage transport until now had remained unclear. Thus, ligand-directed delivery through the selective targeting of α3β1 integrins represents a substantial advance over conventional non-targeted aerosol formulations that require penetration enhancers or solubilizing carriers for drug stability and dispersion (Liang, Z. et al., Drug Discov. Today, 2015, 20:380-389). The present disclosure establishes an internalizing mechanism of a selective ligand by α3β1 integrins in the lung, which is useful, in a non-limiting example, for pulmonary delivery and consequent applied immunization in vivo.

To support the translational application of the ligand peptide-directed pulmonary delivery approach introduced in the present disclosure, a targeted phage display-based protocol was designed in mice and non-human primates as an aerosol strategy for pulmonary and systemic humoral immunization as a proof-of-principle towards lung vaccination against multiple diseases. Given the constant immunogenic exposure to pathogens through the airways, the lung tissue is a highly active site (yet often either unappreciated or underappreciated) of host defense in which efficient antigen presentation takes place. Thus, pulmonary delivery of aerosolized antigens has many advantages over other routes of administration, particularly for the development of candidate vaccines or therapeutics against respiratory infections (including but not limited to SARS-CoV-2). Moreover, the selective pulmonary transport of CAKSMGDIVC-displaying phage particles followed by activation of a specific local and systemic humoral response recapitulated long-held principles of vaccinology. The pulmonary delivery system studied here has unique translation relevance towards the development of vaccines against airborne pathogens.

The translational benefits of aerosol phage-based vaccines are multifaceted. In one aspect, phage particles are highly stable under harsh environmental conditions and their large-scale production is extremely cost-effective if compared to traditional methods used for vaccine production (Barbu, E. M. et al., Cold Spring Harb. Perspect. Biol., 2016, 8(10):α023879; Bao, Q. et al., Adv. Drug Deliv. Rev., 2019, 145:40-56). In another aspect, phage therapies and phage-based vaccines do not induce detectable toxic side effects. In fact, phage particles have been used as antibiotics against multidrug-resistant bacteria, or as immunogenic vaccine carriers for nearly a century and it has been proven safe and effective (Schmidt, C., Nat. Biotechnol., 2019, 37:581-586; Barbu, E. M. etal., Cold Spring Harb. Perspect. Biol., 2016, 8(10):α023879). Indeed, phage administration has been leveraged towards discovery and transgene delivery applications, including the administration of phage libraries in mice, pet dogs, non-human primates, and even patients (Staquicini, F. I. et al., J. Clin. Invest., 2011, 121:161-173; Pasqualini, R. etal., Nature, 1996, 380:364-366; Hajitou, A. et al., Cell, 2006, 125:385-398; Arap, W. et al., Nat. Med., 2002, 8:121-127). In yet another aspect, because native phage particles have no tropism toward mammalian cells and do not replicate inside eukaryotic cells, their use is generally considered safe when compared to other classic viral-based vaccination strategies. In yet another aspect, unlike conventional peptide-based vaccines that may often become inactivated due to minimal temperature excursions (˜1° C.), the system introduced here has no cumbersome and expensive requirements for keeping a stringent so-called “cold-chain” during field applications, particularly in the developing world. In yet another aspect, the ligand-receptor discovery and vaccination properties of the disclosed phage display-based system can also be used for the development of targeted pulmonary delivery of phage chimeras displaying other viral antigens, or entire transgenes by using a hybrid vector of adeno-associated virus (AAV) and phage (termed AAVP). In yet another aspect, phage particles have been used as immunogenic vaccine carriers for decades as themselves are very strong immunogens, serving as a potent adjuvant to elicit sustained humoral responses (Trepel, M. et al., Cancer Res., 2001, 61:8110-8112; de la Cruz, V. F. et al., J. Biol. Chem., 1988, 263:4318-4322; Aghebati-Maleki, L. et al., J. Biomed. Sci., 2016, 23:66; Barbu, E. M. et al., Cold Spring Harb. Perspect. Biol., 2016, 8(10):α023879). Indeed, in certain embodiments, phage particles as antibacterial agents in the setting of multidrug resistant bacterial infections or the ongoing pandemic of SARS-CoV-2 coronavirus can be well suited for use within the targeted aerosol strategy described herein.

### ENUMERATED EMBODIMENTS

The following enumerated embodiments are provided, the numbering of which is not to be construed as designating levels of importance.

Embodiment 1 provides an isolated transport peptide comprising at least one amino acid sequence selected from the group consisting of CAINSLSRKC (SEQ ID NO:1), CAKSMGDIVC (SEQ ID NO:2), CGRKQVESSC (SEQ ID NO:3), CRGKSAEGTC (SEQ ID NO:4), AINSLSRK (SEQ ID NO:5), AKSMGDIV (SEQ ID NO:6), GRKQVESS (SEQ ID NO:7), and/or RGKSAEGT (SEQ ID NO:8).

Embodiment 2 provides the transport peptide of Embodiment 1, which comprises the amino acid sequence of SEQ ID NO:2.

Embodiment 3 provides the transport peptide of any one of Embodiments 1-2, which consists essentially of an amino acid sequence selected from the group consisting of SEQ ID NOs:1-4.

Embodiment 4 provides the transport peptide of any one of Embodiments 1-3, which consists of an amino acid sequence selected from the group consisting of SEQ ID NOs:1-4.

Embodiment 5 provides a solid particle, wherein the surface of the solid particle displays the transport peptide of any one of Embodiments 1-4, wherein the solid particle is selected from the group consisting of a bacteriophage, engineered cell, tissue fragment, nanoparticle, vesicle, dendrimer, virus-like particle, adenovirus, adeno-associated virus (AAV), adeno-associated virus phage (termed AAVP), and any combinations thereof.

Embodiment 6 provides the solid particle of Embodiment 5, wherein the transport peptide is attached to or displayed on the surface of the solid particle.

Embodiment 7 provides the solid particle of any one of Embodiments 5-6, wherein the transport peptide is attached to or displayed on at least a fraction of the surface of the solid particle.

Embodiment 8 provides the solid particle of any one of Embodiments 5-7, wherein the solid particle is a filamentous phage.

Embodiment 9 provides the solid particle of any one of Embodiments 5-8, which further comprises an agent selected from the group consisting of a therapeutic agent, biologically active molecule, imaging agent, radioactive agent, salt, peptide, protein, lipid, nucleic acid, gas, and any combinations thereof, wherein the agent is attached to and/or contained within the solid particle.

Embodiment 10 provides the solid particle of any one of Embodiments 5-9, wherein the solid particle is a filamentous phage.

Embodiment 11 provides the solid particle of Embodiment 10, wherein the filamentous bacteriophage comprises a fd, fl, or M13 bacteriophage.

Embodiment 12 provides the solid particle of any one of Embodiments 10-11, wherein the solid particle is a filamentous phage and wherein the surface of the solid particle displays an antigen.

Embodiment 13 provides a method of promoting and/or increasing transport of a solid particle across the air-blood barrier in the lung of a subject, wherein the method comprises administering to the subject the solid particle of any one of Embodiments 5-12, wherein the administration is through a route comprising nasal, buccal, inhalational, intratracheal, intrapulmonary, or intrabronchial.

Embodiment 14 provides a method of promoting systemic circulation of a solid particle in a subject, wherein the method comprises administering to the subject the solid particle of any one of Embodiments 5-12, wherein the administration is through a route comprising nasal, buccal, inhalational, intratracheal, intrapulmonary, or intrabronchial.

Embodiment 15 provides a method of immunizing a subject against a disease or disorder, wherein the method comprises administering to the subject the solid particle of any one of Embodiments 5-12, wherein the surface of the solid particle is further derivatized with an antigen that promotes immune response to the disease or disorder in the subject, wherein the administration is through a route comprising nasal, buccal, inhalational, intratracheal, intrapulmonary, or intrabronchial.

Embodiment 16 provides a method of treating, ameliorating, and/or preventing a disease or disorder in a subject, wherein the method comprises administering to the subject the solid particle of any one of Embodiments 5-12, wherein the surface of the solid particle is further derivatized with an antigen that promotes immune response to the disease or disorder in the subject, wherein the administration is through a route comprising nasal, buccal, inhalational, intratracheal, intrapulmonary, or intrabronchial.

Embodiment 17 provides a method of treating a subject at risk of developing a disease or disorder, wherein the method comprises administering to the subject the solid particle of any one of Embodiments 5-12, wherein the surface of the solid particle is further derivatized with an antigen that promotes immune response to the disease or disorder in the subject, wherein the administration is through a route comprising nasal, buccal, inhalational, intratracheal, intrapulmonary, or intrabronchial.

Embodiment 18 provides the method of any one of Embodiments 13-17, wherein the peptide comprises the amino acid sequence of SEQ ID NO:2.

Embodiment 19 provides the method of any one of Embodiments 13-18, wherein the peptide consists of the amino acid sequence of SEQ ID NO:2.

Embodiment 20 provides the method of any one of Embodiments 13-19, wherein the solid particle is a filamentous phage.

Embodiment 21 provides the method of Embodiment 20, wherein the filamentous bacteriophage comprises a fd, fl, or M13 bacteriophage.

Embodiment 22 provides the method of any one of Embodiments 13-21, wherein the solid particle is administered to the subject in a composition further comprising an immunogenic adjuvant.

Embodiment 23 provides the method of any one of Embodiments 13-22, wherein the subject is a mammal.

Embodiment 24 provides the method of any one of Embodiments 13-23, wherein the subject is a human.

Embodiment 25 provides a vaccine comprising a solid particle, wherein the surface of the solid particle displays at least one transport peptide of claim 1 and wherein the solid particle is selected from the group consisting of a bacteriophage, engineered cell, tissue fragment, nanoparticle, vesicle, dendrimer, virus-like particle (VLP), adenovirus, adeno-associated virus (AAV), adeno-associated virus phage (termed AAVP), and a combination thereof.

Embodiment 26 provides the vaccine of Embodiment 25, wherein the vaccine is selected from the group consisting of a DNA vaccine, an RNA vaccine, a replicating viral vector vaccine, a non-replicating viral vector vaccine, an inactivated viral vector vaccine, a virus-like particle vaccine, and any combination thereof

Embodiment 27 provides the vaccine of any one of Embodiments 25-26, wherein the particle comprises a vaccine active agent selected from the group consisting of a DNA, an RNA, a replicating viral vector, a non-replicating viral vector, an inactivated viral vector, a virus-like particle, and any combination thereof

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof

The disclosures of each and every patent, patent application, and publication cited herein are hereby incorporated herein by reference in their entirety. While this disclosure has been disclosed with reference to specific embodiments, it is apparent that other embodiments and variations of this disclosure can be devised by others skilled in the art without departing from the true spirit and scope of the disclosure. The appended claims are intended to be construed to include all such embodiments and equivalent variations.

1. A transport peptide comprising at least one amino acid sequence selected from the group consisting of CAINSLSRKC (SEQ ID NO:1), CAKSMGDIVC (SEQ ID NO:2), CGRKQVESSC (SEQ ID NO:3), CRGKSAEGTC (SEQ ID NO:4), AINSLSRK (SEQ ID NO:5), AKSMGDIV (SEQ ID NO:6), GRKQVESS (SEQ ID NO:7), and/or RGKSAEGT (SEQ ID NO:8).

2. The transport peptide of claim 1, which comprises the amino acid sequence of SEQ ID NO:2.

3. The transport peptide of claim 1, which consists essentially of an amino acid sequence selected from the group consisting of SEQ ID NOs:1-4.

4. The transport peptide of claim 3, which consists of an amino acid sequence selected from the group consisting of SEQ ID NOs:1-4.

5. A solid particle, wherein the surface of the solid particle displays the transport peptide of claim 1, wherein the solid particle is selected from the group consisting of a bacteriophage, engineered cell, tissue fragment, nanoparticle, vesicle, dendrimer, virus-like particle, adenovirus, adeno-associated virus (AAV), adeno-associated virus phage (termed AAVP), and any combinations thereof.

6. The solid particle of claim 5, wherein the transport peptide is attached to or displayed on at least a fraction of the surface of the solid particle.

7. (canceled)

8. The solid particle of claim 5, wherein the solid particle is a filamentous phage and optionally wherein the surface of the solid particle displays an antigen.

9. The solid particle of claim 5, which further comprises an agent selected from the group consisting of a therapeutic agent, biologically active molecule, imaging agent, radioactive agent, salt, peptide, protein, lipid, nucleic acid, gas, and any combinations thereof, wherein the agent is attached to or contained within the solid particle.

10. (canceled)

11. The solid particle of claim 8, wherein the filamentous bacteriophage comprises a fd, fl, or M13 bacteriophage.

12. (canceled)

13. A method of promoting or increasing transport of a solid particle across the air-blood barrier in the lung of a subject, wherein the method comprises administering to the subject the solid particle of claim 5, wherein the administration is through a route comprising nasal, buccal, inhalational, intratracheal, intrapulmonary, or intrabronchial optionally wherein the solid particle is administered to the subject in a composition further comprising an immunogenic adjuvant.

14. A method of promoting systemic circulation of a solid particle in a subject, wherein the method comprises administering to the subject the solid particle of claim 5, wherein the administration is through a route comprising nasal, buccal, inhalational, intratracheal, intrapulmonary, or intrabronchial, optionally wherein the solid particle is administered to the subject in a composition further comprising an immunogenic adjuvant.

15. A method of immunizing a subject against a disease or disorder, wherein the method comprises administering to the subject the solid particle of claim 5, wherein the surface of the solid particle is further derivatized with an antigen that promotes immune response to the disease or disorder in the subject, wherein the administration is through a route comprising nasal, buccal, inhalational, intratracheal, intrapulmonary, or intrabronchial optionally wherein the solid particle is administered to the subject in a composition further comprising an immunogenic adjuvant.

16. A method of treating, ameliorating, or preventing a disease or disorder in a subject, wherein the method comprises administering to the subject the solid particle of claim 5, wherein the surface of the solid particle is further derivatized with an antigen that promotes immune response to the disease or disorder in the subject, wherein the administration is through a route comprising nasal, buccal, inhalational, intratracheal, intrapulmonary, or intrabronchial, optionally wherein the solid particle is administered to the subject in a composition further comprising an immunogenic adjuvant.

17. A method of treating a subject at risk of developing a disease or disorder, wherein the method comprises administering to the subject the solid particle of claim 5, wherein the surface of the solid particle is further derivatized with an antigen that promotes immune response to the disease or disorder in the subject, wherein the administration is through a route comprising nasal, buccal, inhalational, intratracheal, intrapulmonary, or intrabronchial, optionally wherein the solid particle is administered to the subject in a composition further comprising an immunogenic adjuvant.

18. The method of claim 13, wherein the peptide comprises the amino acid sequence of SEQ ID NO:2, optionally wherein the peptide consists of the amino acid sequence of SEQ ID NO:2.

19. (canceled)

20. The method of claim 13, wherein the solid particle is a filamentous phage, optionally wherein the filamentous bacteriophage comprises a fd, fl, or M13 bacteriophage.

21. (canceled)

22. (canceled)

23. The method of claim 13, wherein the subject is a mammal, optionally wherein the subject is a human.

24. (canceled)

25. A vaccine comprising a solid particle, wherein the surface of the solid particle displays at least one transport peptide of claim 1 and wherein the solid particle is selected from the group consisting of a bacteriophage, engineered cell, tissue fragment, nanoparticle, vesicle, dendrimer, virus-like particle (VLP), adenovirus, adeno-associated virus (AAV), adeno-associated virus phage (termed AAVP), and any combination thereof.

26. The vaccine of claim 25, wherein the vaccine is selected from the group consisting of a DNA vaccine, an RNA vaccine, a replicating viral vector vaccine, a non-replicating viral vector vaccine, an inactivated viral vector vaccine, a virus-like particle vaccine, and any combination thereof

27. The vaccine of claim 25, wherein the particle comprises a vaccine active agent selected from the group consisting of a DNA, an RNA, a replicating viral vector, a non-replicating viral vector, an inactivated viral vector, a virus-like particle, and any combination thereof.


SEQUENCE LISTING (11 sequences)

SEQ ID NO: 1 - protein, 10 aa, Artificial Sequence
  chemically synthesized
CAINSLSRKC

SEQ ID NO: 2 - protein, 10 aa, Artificial Sequence
  chemically synthesized
CAKSMGDIVC

SEQ ID NO: 3 - protein, 10 aa, Artificial Sequence
  chemically synthesized
CGRKQVESSC

SEQ ID NO: 4 - protein, 10 aa, Artificial Sequence
  chemically synthesized
CRGKSAEGTC

SEQ ID NO: 5 - protein, 8 aa, Artificial Sequence
  chemically synthesized
AINSLSRK

SEQ ID NO: 6 - protein, 8 aa, Artificial Sequence
  chemically synthesized
AKSMGDIV

SEQ ID NO: 7 - protein, 8 aa, Artificial Sequence
  chemically synthesized
GRKQVESS

SEQ ID NO: 8 - protein, 8 aa, Artificial Sequence
  chemically synthesized
RGKSAEGT

SEQ ID NO: 9 - DNA, 20 bp, Artificial Sequence
  chemically synthesized
tgaggtggtatcggcaatga

SEQ ID NO: 10 - DNA, 22 bp, Artificial Sequence
  chemically synthesized
ggatgctgtatttaggccgttt

SEQ ID NO: 11 - protein, 9 aa, Artificial Sequence
  chemically synthesized
CGRRAGGSC